SEQUENCE LISTING (2 sequences)

SEQ ID NO: 1 - protein, 471 aa, Human papillomavirus type 16
MSLWLPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGAVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHTPPAPKEDPLKKYTFWEVNLKEKFSADLDQFPLGRKFLLQ

SEQ ID NO: 2 - protein, 472 aa, Human papillomavirus type 18
MALWRPSDNTVYLPPPSVARVVNTDDYVTRTSIFYHAGSSRLLTVGNPYFRVPAGGGNKQDIPKVSAYQYRVFRVQLPDPNKFGLPDNSIYNPETQRLVWACVGVEIGRGQPLGVGLSGHPFYNKLDDTESSHAATSNVSEDVRDNVSVDYKQTQLCILGCAPAIGEHWAKGTACKSRPLSQGDCPPLELKNTVLEDGDMVDTGYGAMDFSTLQDTKCEVPLDICQSICKYPDYLQMSADPYGDSMFFCLRREQLFARHFWNRAGTMGDTVPPSLYIKGTGMRASPGSCVYSPSPSGSIVTSDSQLFNKPYWLHKAQGHNNGVCWHNQLFVTVVDTTRSTNLTICASTQSPVPGQYDATKFKQYSRHVEEYDLQFIFQLCTITLTADVMSYIHSMNSSILEDWNFGVPPPPTTSLVDTYRFVQSVAITCQKDAAPAENKDPYDKLKFWNVDLKEKFSLDLDQYPLGRKFLVQ

(19) United States
(12) Patent Application Publication
(10) Pub. No.: US 2015/0110824 A1
(43) Pub. Date: Apr. 23, 2015

(54) METHOD OF VACCINATION AGAINST HUMAN PAPILLOMAVIRUS

(71) Applicant: GLAXOSMITHKLINE BIOLOGICALS, SA, Rixensart (BE)

(72) Inventors: Brigitte Desiree Alberte Colau, Rixensart (BE); Sandra Giannini, Rixensart (BE); Laurence Lockman, Wavre (BE)

(21) Appl. No.: 14/385,813

(22) PCT Filed: Mar. 18, 2013
(86) PCT No.: PCT/EP2013/055582

§ 371 Date: Sep. 17, 2014

Related U.S. Application Data
(60) Provisional application No. 61/612,345, filed on Mar. 18, 2012.

(51) Int. Cl. A61K 39/39; C12N 7/00; A61K 39/12

(52) U.S. Cl. CPC A61K 39/39; A61K 39/12; C12N 7/00; C12N 2710/20071; A61K 2039/55572
USPC 424/186.1

(57) ABSTRACT

The disclosure provides immunogenic compositions comprising HPV VLPs from one or more HPV types in combination with an adjuvant comprising a TLR agonist for use in a method for the prevention of HPV infection or disease in an individual, wherein a first dose of the immunogenic composition comprising HPV VLPs and a TLR agonist, is administered followed by a second dose of an immunogenic composition comprising HPV VLPs from one or more HPV types but which does not comprise a TLR agonist.

### BACKGROUND

The present disclosure relates to the field of human vaccines. More particularly, the present disclosure relates to pharmaceutical and immunogenic compositions, for the prevention or treatment of human papillomavirus (HPV) infection or disease, and to methods for vaccination against HPV infection or disease.

Papillomaviruses are small, highly species specific, DNA tumour viruses. Human papillomaviruses are DNA viruses that infect basal epithelial (skin or mucosal) cells. Over 100 individual human papillomavirus (HPV) genotypes have been described. HPVs are generally specific either for the squamous epithelium of the skin (e.g. HPV-1 and -2) or mucosal surfaces (e.g. HPV-6 and -11) and usually cause benign tumours (warts) that persist for several months or years.

Persistent infection with an oncogenic human papillomavirus (HPV) type is a necessary cause of cervical cancer, the second most common cause of cancer deaths among women worldwide. There is international consensus that “high-risk” genotypes, including genotypes 16, 18, 31, 33, 35, 39, 45, 51, 52, 56, 58, 59, 66, 68 and 73 can lead to cervical cancer and are associated with other mucosal anogenital and head and neck cancers. Globally, HPV-16 and HPV-18 are the predominant oncogenic types, cumulatively accounting for over 70-80% of all invasive cervical cancer cases.

Infections with other genotypes, termed “low-risk,” can cause benign or low-grade cervical tissue changes and genital warts (condyloma acuminata), which are growths on the cervix, vagina, vulva and anus in women and the penis, scrotum or anus in men. They also cause epithelial growths over the vocal cords of children and adults (juvenile respiratory papillomatosis or recurrent respiratory papillomatosis) that require surgical intervention.

Two prophylactic HPV vaccines have recently been licensed in many countries. Both use virus-like particles (VLPs) comprised of recombinant L1 capsid proteins of individual HPV types to prevent HPV-16 and -18 cervical precancerous lesions and cancers. Cervarix™ (GlaxoSmithKline Biologicals) contains HPV-16 and -18 VLPs produced in a Trichoplusia ni insect cell substrate using a baculovirus expression vector system and formulated with the immunostimulant 3-O-desacyl-4′-monophosphoryl lipid A (3D MPL, also known as MPL) and aluminium hydroxide salt. Gardasil™ (Merck) contains HPV-16 and -18 VLPs produced in the yeast Saccharomyces cerevisiae and formulated with amorphous aluminium hydroxyphosphate sulphate salt. In addition, Gardasil™ contains VLPs from non-oncogenic types HPV-6 and -11, which are implicated in 75-90% of genital warts. For both vaccines, specific protection against infection with oncogenic types HPV-16 and HPV-18 and associated precancerous lesions has been demonstrated in randomised clinical trials.

The list of oncogenic HPV types which are responsible for causing cervical cancer includes at least HPV types 16, 18, 31, 33, 35, 39, 45, 51, 52, 56, 58, 59, 66, 68 and 73 found in cervical cancer (Mandavi et al, 2005; Quint et al., 2006).

The existing vaccines are able to provide specific protection against infection and/or disease by some of these HPV types and to varying degrees. For example Cervarix™ provides cross protective efficacy against HPV types 33, 31, 45 and 51. HPV-16/18 and these four types cause about 85% of cervical cancer; moreover, there is a particularly high risk of HPV-33 infections progressing to cervical lesions, and HPV-45 is over-represented in adenocarcinoma (Wheeler et al, 2012). However it would be potentially beneficial to provide the high degree of protection against cervical cancer achieved by Cervarix™ and also to provide some protection against infection or disease caused by other HPV types. It would be potentially beneficial to provide a high degree of protection against cervical cancer and also to provide improved protection against genital warts caused by HPV-6 and HPV-11 than is provided by the existing vaccines.

It has now been discovered that by administering one or more doses of an HPV vaccine comprising the adjuvant MPL, in a vaccination scheme with a different HPV vaccine not containing the MPL adjuvant, certain advantages can be achieved. For example, the immune response to certain HPV types present in the vaccine, such as HVP 18, can be increased compared to a vaccination scheme using only aduminium adjuvant. This is seen particularly, but not exclusively, when the MPL containing vaccine is administered first. Alternatively or additionally the cross reactive immune response to certain HPV types not present in the MPL adjuvanted vaccine but present in the aluminium adjuvanted vaccine can be equalled or increased compared to vaccination using only the aluminium adjuvanted vaccine, by administering the MPL containing vaccine first followed by the aluminium adjuvanted vaccine.

### BRIEF SUMMARY

The present disclosure relates to the use of TLR agonist containing HPV vaccines to enhance vaccination against HPV. The disclosure further relates to using different HPV vaccines, including a TLR agonist containing vaccine, in a particular sequence in a vaccination scheme. In particular the disclosure relates to improving the response to certain HPV types by the use of a TLR agonist containing HPV vaccine in a vaccination scheme employing a non-TLR agonist containing HPV vaccine. The disclosure further relates to a vaccination scheme which employs a priming vaccine which induces a cross reactive immune response against one or more HPV types absent from the priming vaccine, followed by a boosting vaccine which contains one or more HPV types absent from the priming vaccine and to which a cross reactive response has been induced by the priming vaccine. The immune response to the absent HPV types is boosted by the boosting vaccine to a level which is at least equal to and may be higher than the immune response induced by an equivalent number of doses of the boosting vaccine alone. The use of different priming and boosting vaccines also enables the use of different vaccines in a vaccination schedule.

In one aspect the invention provides a first immunogenic composition comprising HPV VLPs from one or more HPV types in combination with an adjuvant comprising a TLR agonist for use in a method for the prevention of HPV infection or disease in an individual, which method comprises:

(i) administering to the individual at least one dose of the first immunogenic composition; followed by

(ii) administering to the individual at least one dose of a second immunogenic composition comprising HPV VLPs from one or more HPV types which second immunogenic composition does not comprise a TLR agonist;

wherein the first immunogenic composition increases at least one of a type specific immune response or cross reactive immune response to an HPV type present in the second immunogenic composition, which is not present in the first immunogenic composition.

In a further aspect the invention provides an immunogenic composition comprising HPV VLPs from at least one HPV type in combination with an adjuvant comprising an aluminium salt without a TLR4 agonist, for use in a method for the prevention of HPV infection or disease in an individual, which method comprises:

(i) administering to the individual at least one dose of a first immunogenic composition comprising HPV VLPs from one or more HPV types in combination with an adjuvant comprising a TLR agonist; and

(ii) administering to the individual at least one dose of a second immunogenic composition which is the immunogenic composition comprising HPV VLPs in combination with an aluminium salt without a TLR4 agonist;

wherein the first immunogenic composition increases at least one of a type specific immune response or cross reactive immune response to an HPV type present in the second immunogenic composition, which is not present in the first immunogenic composition.

In another aspect the invention provides a method for the prevention of HPV infection or disease in an individual, which method comprises:

(i) administering to the individual at least one dose of a first immunogenic composition comprising HPV VLPs from one or more HPV types in combination with an adjuvant comprising a TLR agonist; and

(ii) administering to the individual at least one dose of a second immunogenic composition comprising HPV VLPs from one or more HPV types which second immunogenic composition does not comprise a TLR agonist;

wherein the first immunogenic composition increases at least one of a type specific immune response or cross-reactive immune response to a type present in the second immunogenic composition, which is not present in the first immunogenic composition.

In another aspect the invention provides a kit comprising:
(i) a first immunogenic composition comprising VLPs from at least one HPV type in combination with an adjuvant comprising a TLR agonist; and
(ii) a second immunogenic composition comprising VLPs from at least one HPV type and which does not comprise a TLR agonist.

In another aspect the invention provides a method for inducing antibodies against HPV in humans comprising administering to a human first and second immunogenic compositions described herein.

In another aspect the invention provides a method for inducing neutralising antibodies against HPV in humans comprising administering to a human first and second immunogenic compositions described herein. Such a method can also induce cross neutralising antibodies.

In another aspect the invention provides a method for inducing cellular immunity against HPV in humans comprising administering to a human first and second immunogenic compositions described herein.

In another aspect the invention provides a method for inducing neutralising antibodies and cellular immunity against HPV in humans comprising administering to a human first and second immunogenic compositions described herein. Such a method can also induce cross neutralising antibodies.

In a further aspect the disclosure relates to a first immunogenic composition comprising HPV VLPs from one or more HPV types in combination with an adjuvant comprising a TLR agonist, for use in a method for enhancing the prevention of HPV infection or disease, wherein the method comprises administering one or more doses of the immunogenic composition to an individual who has already received one or more doses of a second immunogenic composition comprising HPV VLPs from one or more HPV types but which does not comprise a TLR agonist.

### BRIEF DESCRIPTION OF THE DRAWINGS

FIGS. 1-20 and 22-33 show total and neutralising antibody responses in mice, measured by ELISA and psuedovirus neutralisation assay respectively, in mice following immunisation with different vaccination schemes with Cervarix™ and Gardasil™. These are the results of three separate experiments, Example 1 data grouped as FIGS. 1-16, Example 2 data as FIGS. 17-20 and Example 3 data as FIGS. 22-33. FIG. 21 shows the results of a protection assay which formed part of Example 2 and FIGS. 34-38 show the results of a protection assay which formed part of Example 3.

Further details are as follows:

FIG. 1 shows total anti-HPV 16 L1 VLP antibody responses.

FIG. 2 shows a summary of statistical analysis for total anti-HPV-16 responses.

FIG. 3 shows neutralising anti-HPV-16 L1 VLP antibody responses.

FIG. 4 shows a summary of statistical analysis for neutralising anti-HPV 16 responses.

FIG. 5 shows total anti-HPV 18 L1 VLP antibody responses.

FIG. 6 shows a summary of statistical analysis for total anti-HPV-18 responses.

FIG. 7 shows neutralising anti-HPV-18 L1 VLP antibody responses.

FIG. 8 shows a summary of statistical analysis for neutralising anti-HPV 18 responses.

FIG. 9 shows total anti-HPV-6 L1 VLP antibody responses.

FIG. 10 shows a summary of statistical analysis for total anti-HPV6 antibody responses.

FIG. 11 shows neutralising anti-HPV-6 L1 VLP antibody responses.

FIG. 12 shows a summary of statistical analysis for neutralising anti-HPV6 antibody responses.

FIG. 13 shows total anti-HPV-11 L1 VLP antibody responses.

FIG. 14 shows a summary of statistical analysis for total anti-HPV11 antibody responses.

FIG. 15 shows neutralising anti-HPV-11 L1 VLP antibody responses.

FIG. 16 shows a summary of statistical analysis for neutralising anti-HPV11 antibody responses.

FIG. 17 shows total anti-HPV-18 antibody responses (Example 2).

FIG. 18 shows neutralizing anti-HPV-18 antibody responses (Example 2).

FIG. 19 shows total anti-HPV-11 antibody responses (Example 2).

FIG. 20 shows neutralizing anti-HPV-11 antibody responses (Example 2).

FIG. 21 shows comparative protection percentages and bioluminescent signals at 1 month post II in mice following intravaginal challenge experiment in Example 2.

FIG. 22 shows total anti-HPV-18 L1 VLP antibodies at 1M PIII (Example 3).

FIG. 23 shows total anti-HPV-18 L1 VLP antibodies at 6M PIII (Example 3).

FIG. 24 shows neutralizing anti-HPV-18 L1 VLP antibodies at 1M PIII (Example 3).

FIG. 25 shows neutralizing anti-HPV-18 L1 VLP antibodies at 6M PIII (Example 3).

FIG. 26 shows total anti-HPV-6 L1 VLP antibodies at 1M PIII (Example 3).

FIG. 27 shows total anti-HPV-6 L1 VLP antibodies at 6M PIII (Example 3).

FIG. 28 shows neutralizing anti-HPV-6 L1 VLP antibodies at 1M PIII (Example 3).

FIG. 29 shows neutralizing anti-HPV-6 L1 VLP antibodies at 6M PIII (Example 3).

FIG. 30 shows total anti-HPV-11 L1 VLP antibodies at 1M PIII (Example 3).

FIG. 31 shows total anti-HPV-11 L1 VLP antibodies at 6M PIII (Example 3).

FIG. 32 shows neutralizing anti-HPV-11 L1 VLP antibodies at 1M PIII (Example 3).

FIG. 33 shows neutralizing anti-HPV-11 L1 VLP antibodies at 6M PIII (Example 3).

FIG. 34 shows comparative protection percentages and bioluminescent signals (radiance, Ph/Sec/cm²) at 6M post III (Example 3).

FIG. 35 shows comparative protection percentages and bioluminescent signals (radiance, Ph/Sec/cm²) at 1M post III (Example 3).

FIG. 36 shows comparative protection percentages and bioluminescent signals (radiance, Ph/Sec/cm²) at 6M post III (Example 3).

FIG. 37 shows comparative protection percentages and bioluminescent signals (radiance, Ph/Sec/cm²) at 1M post III (E

[symbol]

FIG. 38 shows comparative protection percentages and bioluminescent signals (radiance, Ph/Sec/cm²) at 6M post III (Example 3).

### DETAILED DESCRIPTION

The invention describes for the first time the use of a TLR agonist-containing HPV vaccine in individuals also receiving a non TLR agonist-containing HPV vaccine, to increase the immune response to one or more HPV types present in the vaccines, in particular high risk HPV types for cervical cancer or low risk HPV types causing genital warts. The invention further describes the use of a TLR agonist-containing HPV vaccine to generate a cross reactive immune response to an HPV type administered in a second, non TLR agonist-containing vaccine. More particularly the invention describes a method for the prevention of HPV related disease or infection by administering different priming and boosting vaccines and wherein the priming vaccine induces an immune response against an HPV type not present in the priming vaccine but which is present in the boosting vaccine. The invention offers the possibility of substituting one vaccine for another in a vaccine schedule without reducing the immune response to HPV types absent from one of the vaccines and more importantly while improving the immune response to certain HPV types.

In one embodiment, the first immunogenic composition comprises HPV 16 and/or HPV 18 VLPs. In a particular embodiment the first immunogenic composition comprises only HPV 16 and HPV 18 VLPs and no other HPV VLPs.

In one embodiment the first immunogenic composition increases the type specific immune response to HPV 16 or HPV 18 or both HPV 16 and HPV 18.

The increase in type specific immune response may be an increase in the immune response when compared to the immune response to the particular HPV type when an equivalent number of doses of only the second immunogenic composition i.e. the composition which is not adjuvanted with a TLR, are administered

In one embodiment the first immunogenic composition generates a cross reactive immune response against one or more high risk or low risk HPV types present in the second immunogenic composition.

The so called “high risk” HPV types responsible for cervical cancer are genotypes 16, 18, 31, 33, 35, 39, 45, 51, 52, 56, 58, 59, 66, 68 and 73 but it will be recognised that this list may be added to over time as more HPV types are found. The so-called “low risk” mucosal HPV types are types which have a low risk of causing cancer such as HPV 6 and 11 causing genital warts, types associated with common warts such as HPV 2 and 3 and HPV 76 associated with benign cutaneous warts. In an embodiment the low risk HPV types present in compositions used in the invention are HPV 6 or HPV 11 or HPV 6 and HPV 11.

In one embodiment the first immunogenic composition increases the cross reactive immune response to a type present in the second immunogenic composition, which is not present in the first immunogenic composition, compared to the immune response to that type when an equivalent number of doses of only the second immunogenic composition are administered.

The immune response generated against a particular HPV type can be measured by a suitable assay for specific antibodies to that HPV type, for example an ELISA and/or pseudoneutralisation assay, such as are described herein in the Examples or in Harper et al 2004, Dessy et al 2008 or Pastrana et al 2004.

In one embodiment the second immunogenic composition comprises HPV 6, HPV 11, HPV 16 and HPV 18 VLPs with or without further HPV VLPs. Such further HPV types may include additional high risk oncogenic HPV types such as one or more of HPV 31, HPV 33, HPV 45, HPV 52 and HPV 58, which may be present in any combination. In a particular embodiment HPV 6, 11, 16, 18, 31, 33, 45, 52 and 58 VLPs are present in the second immunogenic composition in a 9-valent HPV vaccine.

As used herein, a priming composition is an immunogenic composition which is administered before a boosting composition.

Similarly a boosting composition is an immunogenic composition which is administered after a priming composition.

The priming and boosting compositions described herein are immunogenic compositions, that is they are compositions of matter suitable for administration to a human or animal subject (e.g., in an experimental setting) that is capable of eliciting a specific immune response, e.g., against a pathogen, such as Human Papillomavirus. As such, an immunogenic composition includes one or more antigens (for example, antigenic subunits of viruses, e.g., polypeptides, thereof) or antigenic epitopes. An immunogenic composition can also include additional components capable of eliciting or enhancing an immune response, such as an excipient, carrier, and/or adjuvant. In certain instances, immunogenic compositions are administered to elicit an immune response that protects the subject against symptoms or conditions induced by a pathogen. In some cases, symptoms or disease caused by a pathogen is prevented (or treated, e.g., reduced or ameliorated) by inhibiting replication of the pathogen (e.g., Human papillomavirus) following exposure of the subject to the pathogen. For example, in the context of this disclosure, certain embodiments of immunogenic compositions that are intended for administration to a subject or population of subjects for the purpose of eliciting a protective or palliative immune response against human papillomavirus are vaccine compositions or vaccines.

The term “vaccine” refers to a composition that comprises an immunogenic component capable of provoking an immune response in an individual, such as a human, wherein the composition optionally contains an adjuvant. A vaccine for HPV suitably elicits a protective immune response against incident infection, or persistent infection, or cytological abnormality such as ASCUS, CIN1, CIN2, CIN3, or cervical cancer caused by one or more HPV types.

A dose of immunogenic composition as described herein may be a human dose. By the term “human dose” is meant a dose which is in a volume suitable for human use. A human dose comprises an amount of antigen suitable for generating an immune response in a human. Generally the volume of a human dose is a liquid between 0.3 and 1.5 ml in volume. In one embodiment, a human dose is 0.5 ml. In a further embodiment, a human dose is higher than 0.5 ml, for example 0.6, 0.7, 0.8, 0.9 or 1 ml. In a further embodiment, a human dose is between 1 ml and 1.5 ml.

An immune response generated by one HPV type against another HPV type is a cross reactive immune response. The existence or not of a cross reactive immune response as described herein can be detected and measured by any suitable assay for measuring specific antibodies to the relevant HPV type in particular to VLPs of the relevant HPV type. Methods for screening antibodies are well known in the art. An ELISA can be used to assess cross reactivity of antibodies, for example an ELISA as described herein in the Examples. A suitable ELISA is also described in Harper et al 2004 (see webappendix). A cross reactive response may also be cross neutralising and antibodies can be test for neutralisation and cross neutralisation properties using a suitable assay such as a pseudovirus neutralisation assay, for example as described herein in the Examples. Suitable pseudovirus neutralisation assays are described in Dessy et al 2008 and Pastrana et al 2004.

The first and second immunogenic compositions described herein typically include at least one pharmaceutically acceptable diluent or carrier and optionally (for the second immunogenic composition) an adjuvant.

An “adjuvant” is an agent that enhances the production of an immune response in a non-specific manner. Common adjuvants include suspensions of minerals (alum, aluminum hydroxide, aluminum phosphate) onto which antigen is adsorbed; emulsions, including water-in-oil, and oil-in-water (and variants therof, including double emulsions and reversible emulsions), liposaccharides, lipopolysaccharides, immunostimulatory nucleic acids (such as CpG oligonucleotides), liposomes, toll-like receptor agonists (particularly, TLR2, TLR4, TLR7/8 and TLR9 agonists), and various combinations of such components.

In one embodiment, the VLPs in either the first or second immunogenic composition, or both, are used in combination with aluminium, and can be adsorbed or partially adsorbed onto aluminium adjuvant for example aluminium hydroxide or amorphous aluminium hydroxyphosphate sulphate.

In one embodiment the TLR agonist in the first immunogenic composition is a non-toxic derivative of lipid A, such as monophosphoryl lipid A or more particularly 3-O-desacyl-4′-monophoshoryl lipid A (3D-MPL), or QS21. In one embodiment the MPL is used in combination with aluminium hydroxide.

In one embodiment the second immunogenic composition comprises an aluminium salt for example amorphous aluminum hydroxyphosphate sulphate.

When VLPs are adsorbed on to aluminium containing adjuvants, the VLPs can be adsorbed to the aluminium adjuvant prior to mixing of the VLPs to form the final vaccine product.

Thus, in one embodiment the priming composition comprises an aluminium salt. The VLPs may be adsorbed or partially adsorbed onto the aluminium salt. In a particular embodiment the adjuvant is aluminium hydroxide and 3D MPL. Compositions according to the present disclosure comprising such an adjuvant can be prepared as described for example in WO 00/23105 incorporated herein by reference.

In one embodiment the second immunogenic composition comprises an aluminium salt. The VLPs may be adsorbed or partially adsorbed onto the aluminium salt. In a particular embodiment the aluminium salt is amorphous aluminum hydroxyphosphate sulphate.

In a particular embodiment the first immunogenic composition comprises aluminium hydroxide and 3D MPL and the second immunogenic composition comprises amorphous aluminum hydroxyphosphate sulphate.

In one embodiment the TLR agonist for use with HPV antigens in the first immunogenic composition described herein is a non-toxic bacterial lipopolysaccharide derivative. An example of a suitable non-toxic derivative of lipid A, as already described, is monophosphoryl lipid A or more particularly 3-Deacylated monophoshoryl lipid A (3D-MPL). 3D-MPL is sold under the name MPL by GlaxoSmithKline Biologicals N.A., and is referred throughout the document as MPL or 3D-MPL. See, for example, U.S. Pat. Nos. 4,436,727; 4,877,611; 4,866,034 and 4,912,094. 3D-MPL primarily promotes CD4+ T cell responses with an IFN-γ (Thi) phenotype. 3D-MPL can be produced according to the methods disclosed in GB2220211 A. Chemically it is a mixture of 3-deacylated monophosphoryl lipid A with 3, 4, 5 or 6 acylated chains. In the compositions of the present invention small particle 3D-MPL can be used. Small particle 3D-MPL has a particle size such that it can be sterile-filtered through a 0.22 μm filter. Such preparations are described in WO94/21292.

In other embodiments, the lipopolysaccharide can be a β(1-6) glucosamine disaccharide, as described in U.S. Pat. No. 6,005,099 and EP Patent No. 0 729 473 B1. One of skill in the art would be readily able to produce various lipopolysaccharides, such as 3D-MPL, based on the teachings of these references. In addition to the aforementioned immunostimulants (that are similar in structure to that of LPS or MPL or 3D-MPL), acylated monosaccharide and disaccharide derivatives that are a sub-portion to the above structure of MPL are also suitable adjuvants. In other embodiments, the adjuvant is a synthetic derivative of lipid A, some of which are TLR-4 agonists, and include, but are not limited to:

OM174 (2-deoxy-6-o-[2-deoxy-2-[(R)-3-dodecanoyloxytetra-decanoylamino]-4-o-phosphono-□-D-glucopyranosyl]-2-[(R)-3-hydroxytetradecanoylamino]-β-D-glucopyranosyldihydrogenphosphate), (WO 95/14026)

OM 294 DP (3S,9R)-3-[(R)-dodecanoyloxytetradecanoylamino]-4-oxo-5-aza-9(R)-[(R)-3-hydroxytetradecanoylamino]decan-1,10-diol,1,10-bis(dihydrogenophosphate) (WO 99/64301 and WO 00/0462)

OM 197 MP-Ac DP (3S-,9R)-3-□(R)-dodecanoyloxytetradecanoylamino]-4-oxo-5-aza-9-[(R)-3-hydroxytetradecanoylamino]decan-1,10-diol,1-dihydrogenophosphate 10-(6-aminohexanoate) (WO 01/46127)

Other TLR4 ligands which can be used are alkyl Glucosaminide phosphates (AGPs) such as those disclosed in WO 98/50399 or U.S. Pat. No. 6,303,347 (processes for preparation of AGPs are also disclosed), suitably RC527 or RC529 or pharmaceutically acceptable salts of AGPs as disclosed in U.S. Pat. No. 6,764,840. Some AGPs are TLR4 agonists, and some are TLR4 antagonists. Both are thought to be useful as adjuvants.

Other suitable TLR-4 ligands, capable of causing a signaling response through TLR-4 (Sabroe et al, JI 2003 p 1630-5) are, for example, lipopolysaccharide from gram-negative bacteria and its derivatives, or fragments thereof, in particular a non-toxic derivative of LPS (such as 3D-MPL). Other suitable TLR agonists are: heat shock protein (HSP) 10, 60, 65, 70, 75 or 90; surfactant Protein A, hyaluronan oligosaccharides, heparan sulphate fragments, fibronectin fragments, fibrinogen peptides and b-defensin-2, and muramyl dipeptide (MDP). In one embodiment the TLR agonist is HSP 60, 70 or 90. Other suitable TLR-4 ligands are as described in WO 2003/011223 and in WO 2003/099195, such as compound I, compound II and compound III disclosed on pages 4-5 of WO2003/011223 or on pages 3-4 of WO2003/099195 and in particular those compounds disclosed in WO2003/011223 as ER803022, ER803058, ER803732, ER804053, ER804057, ER804058, ER804059, ER804442, ER804680, and ER804764. For example, one suitable TLR-4 ligand is ER804057.

In one embodiment of the present invention, a TLR agonist is used that is capable of causing a signaling response through TLR-1. Suitably, the TLR agonist capable of causing a signaling response through TLR-1 is selected from: Tri-acylated lipopeptides (LPs); phenol-soluble modulin; Mycobacterium tuberculosis LP; S-(2,3-bis(palmitoyloxy)-(2-RS)-propyl)-N-palmitoyl-(R)-Cys-(S)-Ser-(S)-Lys(4)-OH, trihydrochloride (Pam3Cys) LP which mimics the acetylated amino terminus of a bacterial lipoprotein and OspA LP from Borrelia burgdorfei. In an alternative embodiment, a TLR agonist is used that is capable of causing a signaling response through TLR-2. Suitably, the TLR agonist capable of causing a signaling response through TLR-2 is one or more of a lipoprotein, a peptidoglycan, a bacterial lipopeptide from M tuberculosis, B burgdorferi or T pallidum; peptidoglycans from species including Staphylococcus aureus; lipoteichoic acids, mannuronic acids, Neisseria porins, bacterial fimbriae, Yersina virulence factors, CMV virions, measles haemagglutinin, and zymosan from yeast. In an alternative embodiment, a TLR agonist is used that is capable of causing a signaling response through TLR-3. Suitably, the TLR agonist capable of causing a signaling response through TLR-3 is double stranded RNA (dsRNA), or polyinosinic-polycytidylic acid (Poly IC), a molecular nucleic acid pattern associated with viral infection. In an alternative embodiment, a TLR agonist is used that is capable of causing a signaling response through TLR-5. Suitably, the TLR agonist capable of causing a signaling response through TLR-5 is bacterial flagellin. In an alternative embodiment, a TLR agonist is used that is capable of causing a signaling response through TLR-6. Suitably, the TLR agonist capable of causing a signaling response through TLR-6 is mycobacterial lipoprotein, di-acylated LP, and phenol-soluble modulin. Additional TLR6 agonists are described in WO 2003/043572. In an alternative embodiment, a TLR agonist is used that is capable of causing a signaling response through TLR-7. Suitably, the TLR agonist capable of causing a signaling response through TLR-7 is a single stranded RNA (ssRNA), loxoribine, a guanosine analogue at positions N7 and C8, or an imidazoquinoline compound, or derivative thereof. In one embodiment, the TLR agonist is imiquimod. Further TLR7 agonists are described in WO 2002/085905.

The amount of 3D-MPL used in a dose is suitably able to enhance an immune response to an antigen in a human. In particular a suitable 3D MPL amount is that which improves the immunological potential of the composition compared to the unadjuvanted composition, or compared to the composition adjuvanted with another amount of 3D MPL, whilst being acceptable from a reactogenicity profile. The amount of 3D-MPL in each human dose of vaccine can be for example between 1-200 μg, or between 10-100 μg, or between 20-80 μg for example 25 μg per dose, or between 40-60 μg for example 50 μg per dose.

The immunogenic compositions described herein can also comprise aluminium or an aluminium compound as a stabiliser.

In one embodiment one dose of the first immunogenic composition is administered followed by one or more doses of the second immunogenic composition, for example one or two or three doses of the second immunogenic composition.

In another embodiment two doses of the first immunogenic composition are administered followed by one or more doses of the second immunogenic composition, for example one or two doses of the second immunogenic composition.

In particular embodiments one dose of the first immunogenic composition is administered followed by two doses of the second immunogenic composition, or two doses of the first immunogenic composition are administered followed by one dose of the second immunogenic composition.

In a particular embodiment no more than two doses of the first immunogenic composition are administered.

For a kit as described herein, the number of doses of each composition can be as described for the use or method.

Thus, the method and use and kit described herein may employ a single dose of the first immunogenic composition, or a single dose of the second immunogenic composition, or a single dose of both the first immunogenic composition and the second immunogenic composition.

In one embodiment the first and second immunogenic compositions comprise HPV VLPs in an amount of 20 μg or more per dose. Each dose may contain, for example, 30 μg of each VLP, or 40 μg of each VLP, or 60 μg of each VLP. Different VLPs may be present in the same or different amounts. The first and second immunogenic compositions may comprise different amounts of the same HPV VLP.

In one embodiment the first immunogenic composition comprises HPV 16 and HPV 18 VLPs in an amount of 20 μg per dose.

In one embodiment the second immunogenic composition comprises HPV 6, HPV 11, HPV 16 and HPV 18 VLPs in an amount of 20 μg, 40 μg, 40 μg and 20 μg per dose respectively.

Administration of the immunogenic compositions can follow any schedule for a 2 or 3 or more dose vaccination, for example a 0, 1 month schedule, a 0, 2 month schedule, a 0, 3 month schedule, a 0, 4 month schedule, a 0, 5 month schedule or a 0, 6 month schedule for a 2 dose vaccine; a 0, 1 6 month schedule, a 0, 2, 6 month schedule, a 0, 3, 6 month schedule, a 0, 4, 6 schedule for a 3 dose vaccination. Thus the second dose may be administered for example one month or two months or three months or four months or five months or six months or [symbol] months or up to twenty-four months after the first dose. Similarly a third dose may be administered one month or two months or three months or four months or five months or six months or up to twelve months or up to twenty-four months after the second dose.

HPV VLPs and methods for the production of VLPs are well known in the art. VLPs typically are constructed from the HPV L1 protein of the virus and can also include the L2 protein. See for example WO9420137, U.S. Pat. No. 5,985,610, WO9611272, U.S. Pat. No. 6,599,508B1, U.S. Pat. No. 6,361,778B1, EP595935 for VLPs.

In any of the embodiments described herein the HPV VLPs can comprise HPV L1 protein or an immunogenic fragment thereof, with or without another protein or peptide such as an L2 protein or peptide.

In one embodiment the VLPs in the first immunogenic composition are comprised of HPV L1 protein or immunogenic fragment thereof within which is inserted one or more epitopes of L2, for example such as is described in WO 2010/149752 incorporated herein by reference. In a particular embodiment the first immunogenic composition comprises such HPV L1 VLPs with one or more epitopes of L2 inserted, together with HPV L1 only VLPs, for example a combination of HPV 16 and HPV 18 L1 only VLPs together with HPV L1 VLPs with one or more epitopes of L2 inserted in the L1.

In another embodiment the the VLPs in the first immunogenic composition are L1 only VLPs which are VLPs comprising L1 or an immunogenic fragment thereof without L2.

In one embodiment the VLPs in the second immunogenic composition are L1 only VLPs comprising L1 or an immunogenic fragment thereof without L2.

In one embodiment the VLPs in the first immunogenic composition comprise truncated L1.

In one embodiment the VLPs in the second immunogenic composition comprise full length L1.

Suitable immunogenic fragments of HPV L1 include truncations, deletions, substitution, or insertion mutants of L1. Such immunogenic fragments can be capable of raising an immune response, said immune response being capable of recognising an L1 protein such as L1 in the form of a virus particle or VLP, from the HPV type from which the L1 protein was derived.

Immunogenic L1 fragments that can be used include truncated L1 proteins. In one embodiment the truncation removes a nuclear localisation signal and optionally also removes DNA binding patterns in the L1 C terminal region. In another aspect the truncation is a C terminal truncation. In a further aspect the C terminal truncation removes fewer than 50 amino acids, such as fewer than 40 amino acids. Where the L1 is from HPV 16 then in another aspect the C terminal truncation removes 34 amino acids from the carboxy terminus of the HPV 16 L1. Where the L1 is from HPV 18 then in a further aspect the C terminal truncation removes 35 amino acids from the carboxy terminus of the HPV 18 L1. Thus a truncated L1 protein can be truncated at the C terminal compared to the wild type L1, so as to remove the nuclear localisation signal and optionally also DNA binding patterns, for example by removal of fewer than 50 or fewer than 40 amino acids from the C terminal end of the protein. Examples of such truncated proteins for L1 from HPV 16 and 18 are given below as SEQ ID Nos: 1 and 2. Truncated L1 Proteins are also described in U.S. Pat. No. 6,060,324, U.S. Pat. No. 6,361,778, and U.S. Pat. No. 6,599,508 incorporated herein by reference.

In one embodiment the HPV 16 L1 amino acid sequence is the following sequence:

| (SEQ ID NO: 1) | |
|---|---|
| MSLWLPSEATVYLPPVPVSKVVSTDEYVARTNIYYHAGT | 60 |
| SRLLAVGHPYFPIKKPNNNKI | |
| LVPKVSGLQYRVFRIHLPDPNKFGFPDTSFYNPDTQRLV | 120 |
| WACVGVEVGRGQPLGVGISGH | |
| PLLNKLDDTENASAYAANAGVDNRECISMDYKQTQLCLI | 180 |
| GCKPPIGEHWGKGSPCTNVAV | |
| NPGDCPPLELINTVIQDGDMVDTGFGAMDFTTLQANKSE | 240 |
| VPLDICTSICKYPDYIKMVSE | |
| PYGDSLFFYLRREQMFVRHLFNRAGAVGENVPDDLYIKG | 300 |
| SGSTANLASSNYFPTPSGSMV | |
| TSDAQIFNKPYWLQRAQGHNNGICWGNQLFVTVVDTTRS | 360 |
| TNMSLCAAISTSETTYKNTNF | |
| KEYLRHGEEYDLQFIFQLCKITLTADVMTYIHSMNSTIL | 420 |
| EDWNFGLQPPPGGTLEDTYRF | |
| VTSQAIACQKHTPPAPKEDPLKKYTFWEVNLKEKFSADL | 471 |
| DQFPLGRKFLLQ | |

The HPV 16 L1 sequence can also be that disclosed in WO94/05792 or U.S. Pat. No. 6,649,167, for example, suitably truncated. Suitable truncates are truncated at a position equivalent to that shown above, as assessed by sequence comparison, and using the criteria disclosed herein.

In one embodiment the HPV 18 L1 amino acid sequence is the following sequence:

| (SEQ ID NO: 2) | |
|---|---|
| MALWRPSDNTVYLPPPSVARVVNTDDYVTRTSIFYH [symbol] | 60 |
| YFRVPAGGGNKQ | |
| DIPKVSAYQYRVFRVQLPDPNKFGLPDNSIYNPETQRLV | 120 |
| WACVGVEIGRGQPLGVGLSGH | |
| PFYNKLDDTESSHAATSNVSEDVRDNVSVDYKQTQLCIL | 180 |
| GCAPAIGEHWAKGTACKSRPL | |
| SQGDCPPLELKNTVLEDGDMVDTGYGAMDFSTLQDTKCE | 240 |
| VPLDICQSICKYPDYLQMSAD | |
| PYGDSMFFCLRREQLFARHFWNRAGTMGDTVPPSLYIKG | 300 |
| TGMRASPGSCVYSPSPSGSIV | |
| TSDSQLFNKPYWLHKAQGHNNGVCWHNQLFVTVVDTTRS | 360 |
| TNLTICASTQSPVPGQYDATK | |
| FKQYSRHVEEYDLQFIFQLCTITLTADVMSYIHSMNSSI | 420 |
| LEDWNFGVPPPPTTSLVDTYR | |
| FVQSVAITCQKDAAPAENKDPYDKLKFWNVDLKEKFSLD | 472 |
| LDQYPLGRKFLVQ | |

[symbol]

indicates data missing or illegible when filed

An alternative HPV 18 L1 sequence is disclosed in WO96/29413, which can be suitably truncated. Suitable truncates are truncated at a position equivalent to that shown above, as assessed by sequence comparison, and using the criteria disclosed herein.

In one embodiment the HPV VLPs of the first immunogenic composition are L1 only VLPs comprising truncated L1 and the HPV VLPs of the second immunogenic composition are L1 only VLPs comprising full length L1.

VLPs can be made in any suitable cell substrate such as yeast cells or bacterial cells or insect cells e.g. using a baculovirus system in insect cells such as cells from Trichoplusia ni, and techniques for preparation of VLPs are well known in the art, such as WO9913056, U.S. Pat. No. 6,416,945B1, U.S. Pat. No. 6,261,765B1 and U.S. Pat. No. 6,245,568, and references therein, the entire contents of which are hereby incorporated by reference.

In one embodiment the HPV VLPs in the first immunogenic composition are expressed in insect cells.

In one embodiment the HPV VLPs in the second immunogenic composition are expressed in yeast.

VLPs can be made by disassembly and reassembly techniques. For example, McCarthy et al, 1998 “Quantitative Disassembly and Reassembly of Human Papillomavirus Type 11 Virus like Particles in Vitro” J. Virology 72(1):33-41, describes the disassembly and reassembly of recombinant L1 HPV 11 VLPs purified from insect cells in order to obtain a homogeneous preparation of VLPs. WO99/13056 and U.S. Pat. No. 6,245,568 also describe disassembly/reassembly processes for making HPV VLPs.

In one embodiment HPV VLPS are ma [symbol] d WO99/13056 or U.S. Pat. No. 6,245,568.

Alternatively VLPs can be made by expressing the L1 protein or immunogenic fragment, extracting it from the production system or cell substrate and purifying the protein while it is predominantly in the form of L1 monomers or pentamers (capsomers), and then forming VLPs from the purified protein. In one embodiment, the extraction and/or purification step is carried out in the presence of a reducing agent such as β-mercaptoethanol (BME), to prevent VLP formation. In one embodiment, the process comprises the step of removing the reducing agent such as BME to allow VLPs to spontaneously form.

VLP formation can be assessed by standard techniques such as, for example, electron microscopy and dynamic laser light scattering.

Optionally the immunogenic compositions can also be formulated or co-administered with other, non-HPV antigens. Suitably these non-HPV antigens can provide protection against other diseases, such as sexually transmitted diseases such as herpes simplex virus (HSV). For example the vaccine may comprise gD or a truncate thereof from HSV. In this way the vaccine provides protection against both HPV and HSV.

In one embodiment the immunogenic composition is provided in a liquid vaccine formulation, although the composition can be lyophilised and reconstituted prior to administration.

The immunogenic compositions described herein can be administered by any of a variety of routes such as oral, topical, subcutaneous, musosal (typically intravaginal), intraveneous, intramuscular, intranasal, sublingual, intradermal and via suppository. Intramuscular and intradermal delivery are preferred.

The dosage of the VLPs can vary with the condition, sex, age and weight of the individual, the administration route and HPV of the vaccine. The quantity can also be varied with the number of VLP types. Suitably the delivery is of an amount of VLP suitable to generate an immunologically protective response. Suitably each vaccine dose comprises 1-100 μg of each VLP, suitably at least 5 μg, or at least 10 μg, for example, between 5-50 μg each VLP, most suitably 10-50 μg of each VLP, such as with 5 μg, 6 μg, 10 μg, 15 μg, 20 μg, 40 μg or 50 μg.

The immunogenic compositions described herein can be tested using standard techniques, for example in standard preclinical models, to confirm that the vaccine is immunogenic.

All of the methods and uses and kits described herein may be for use in adolescent girls aged from 9 and older e.g. 10-15, such as 10-13 years. However, older girls above 15 years old and adult women can also be vaccinated. Similarly the vaccine can be administered to younger age groups such as 2-12 year olds. The vaccine can also be administered to women following an abnormal pap smear or after surgery following removal of a lesion caused by HPV, or who are seronegative and DNA negative for HPV cancer types.

In one embodiment the methods and uses and kits described herein are for use in females in one or more of the following age brackets: 9 to 25 years of age, 10 to 25 years of age, 9 to 19 years of age, 10 to 19 years of age, 9 to 14 years of age, 10 to 14 years of age, 15 to 19 years of age, 20 to 25 years of age, 14 years of age or below, 19 years of age or below, 25 years of age or below.

The methods and uses and kits described herein can be used in men or boys.

The teaching of all references in the present application, including patent applications and granted patents, are herein fully incorporated by reference.

### REFERENCES

Dessy F J, Giannini S L, Bougelet C A, Kemp T J, David M P, Poncelet S M, Pinto L A, Wettendorff M A. Correlation between direct ELISA, single epitope-based inhibition ELISA and pseudovirion-based neutralization assay for measuring anti-HPV-16 and anti-HPV-18 antibody response after vaccination with the AS04-adjuvanted HPV-16/18 cervical cancer vaccine. Hum Vaccin. 2008 November-December; 4(6):425-34. Epub 2008 Nov. 11.

Einstein M H et al. Comparison of the immunogenicity and safety of Cervarix™ and Gardasil® human papillomavirus (HPV) cervical cancer vaccines in healthy women aged 18-45 years. Human Vaccines 5:10, 705-719; October 2009.

Harper D M, Franco E L, Wheeler C, Ferris D G, Jenkins D, Schuind A, Zahaf T, Innis B, Naud P, De Carvalho N S, Roteli-Martins C M, Teixeira J, Blatter M M, Korn A P, Quint W, Dubin G. Efficacy of a bivalent L1 virus-like particle vaccine in prevention of infection with human papillomavirus types 16 and 18 in young women: a randomised controlled trial. Lancet 2004 Nov. 13: 364: 1757-65.

Mandavi A, Monk B J. Vaccines against human papillomavirus and cervical cancer: promises and challenges. Oncologist. 2005 August; 10(7):528-38. Review.

Pastrana D V, Buck C B, Pang Y Y, Thompson C D, Castle P E, FitzGerald P C, Krüger Kjaer S, Lowy D R, Schiller J T. Reactivity of human sera in a sensitive, high-throughput pseudovirus-based papillomavirus neutralization assay for HPV16 and HPV18. Virology. 2004 Apr. 10; 321(2):205-16.

Quint W G, Pagliusi S R, Lelie N, de Villiers E M, Wheeler C M; World Health Organization Human Papillomavirus DNA International Collaborative Study Group. Results of the first World Health Organization international collaborative study of detection of human papillomavirus DNA Results of the first World Health Organization international collaborative study of detection of human papillomavirus DNA. J Clin Microbiol. 2006 February; 44(2):571-9.

Wheeler C M, Castellsague X, Garland S M, Szarewski A, Paavonen J, Naud P, Salmeron J, Chow S-N, Apter D, Kitchener H, Teixeira J C, Skinner S R, Jaisamrarn U, Limson G, Romanowski B, Aoki F Y, Schwarz T F, Poppe W A J, Bosch F X, Harper D M, Huh W, Hardt K, Zahaf T, Descamps D, Struyf F, Dubin G, Lehtinen M. Overall efficacy of HPV-16/18 AS04-adjuvanted vaccine against grade 3 or greater cervical intraepithelial neoplasia: 4-year end-of-study analysis of the randomised, double-blind PATRICIA trial. The Lancet Oncology 2012 January; 13(1):89-99. Published online November 2011.

### EXAMPLE

### Example 1

### Three Dose Immunogenicity Study in Mice

BALB/c mice (23 mice per group) received intramuscular injections at days 0, 21 and 120 days for all groups. All doses were 1/10^{th} of the human dose of antigen. Two control groups received 3 injections of Cervarix™ (HPV-16/18 L1 VLPs 2/2 μg+AS04) or Gardasil™ (HPV-16/18/6/11 L1 VLPs 4/2/2/4 μg+Merck Aluminium hydroxyphosphate sulphate (MAA*)) vaccines. Four other additional groups were injected with Cervarix™ at day 0 and Gardasil™ at days 21 and 120; Cervarix™ at days 0 and 21 followed by Gardasil™ at day 120; Gardasil™ at day 0 followed by Cervarix™ at days 21 and 120 or Gardasil™ at days 0 and 21 followed by Cervarix™ at day 120. *MAA=Merck Aluminium hydroxyphosphate sulphate

Blood was collected at days 42 (D21 PII) and 162 (D42 PIII) and analysed for total antibody titers (ELISA) against HPV-16/18/6 and 11 L1 VLPs. Neutralizing antibody titers (PBNA) against HPV-16/18/6 and 11 were also measured at day 162. Based on previous experiments and using an ANOVA-1 way analysis, a sample size of 23 mice was needed to detect a 2-fold difference between 6 groups with a power of 91%.

There were 6 groups of mice as follows:

| Groups | D0 | D21 | D120 |
|---|---|---|---|
| 1 | Cervarix ™ | Cervarix ™ | Cervarix ™ |
| 2 | Gardasil ™ | Gardasil ™ | Gardasil ™ |
| 3 | Cervarix ™ | Gardasil ™ | Gardasil ™ |
| 4 | Cervarix ™ | Cervarix ™ | Gardasil ™ |
| 5 | Gardasil ™ | Cervarix ™ | Cervarix ™ |
| 6 | Gardasil ™ | Gardasil ™ | Cervarix ™ |

Adjuvant formulations ( 1/10 of human dose)

| Formulations | Aluminium | MPL |
|---|---|---|
| Cervarix ™ | 50 μg Al(OH)₃ | 5 μg |
| Gardasil ™ | 22.5 μg MAA* | — |

Results

Humoral responses to HPV-16, 18, 6 and 11 L1 VLPs after injection of different immunization schemes were monitored by the total antibody and neutralizing antibody responses (see methods given at the end of Example 1).

1. HPV-16 L1 VLP Responses

1.1 Total Antibody Response HPV16 (ELISA, Post II and III)

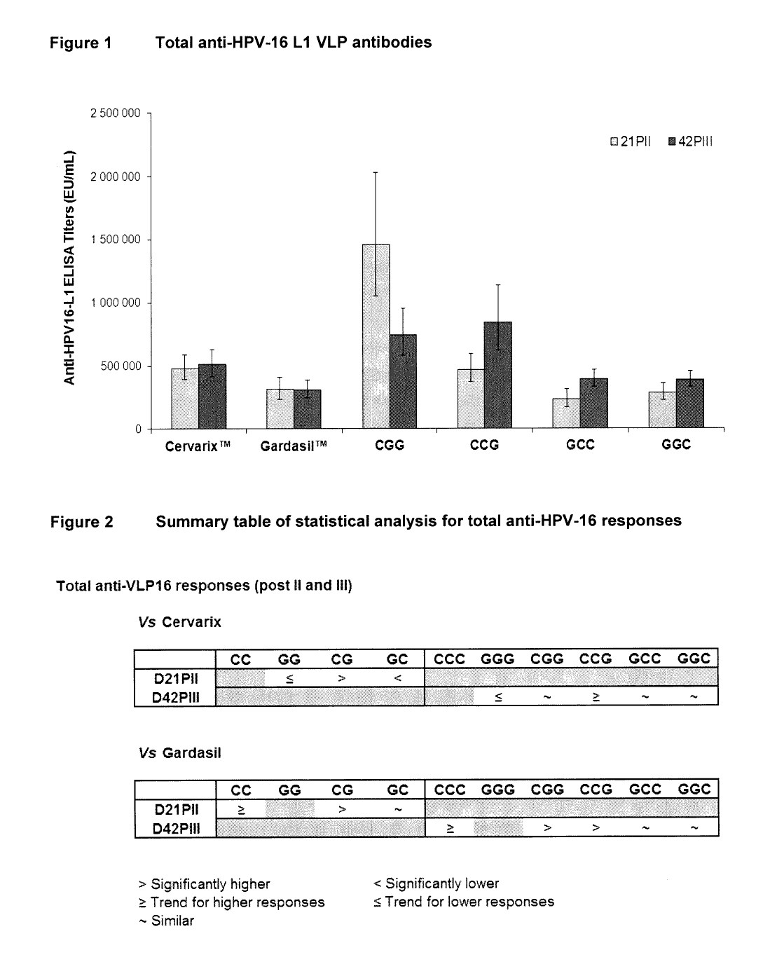

Comparison of total antibody responses (ELISA—see below in Materials and Methods) following immunization with different vaccination schemes is presented in FIG. 1. Summary of statistical analysis comparing all groups to Cervarix™ or Gardasil™ control groups is presented in FIG. 2. Note syringes in the figures correspond to the injection timepoint.

1.2 Neutralization Response HPV16 (PBNA, D42 PIII)

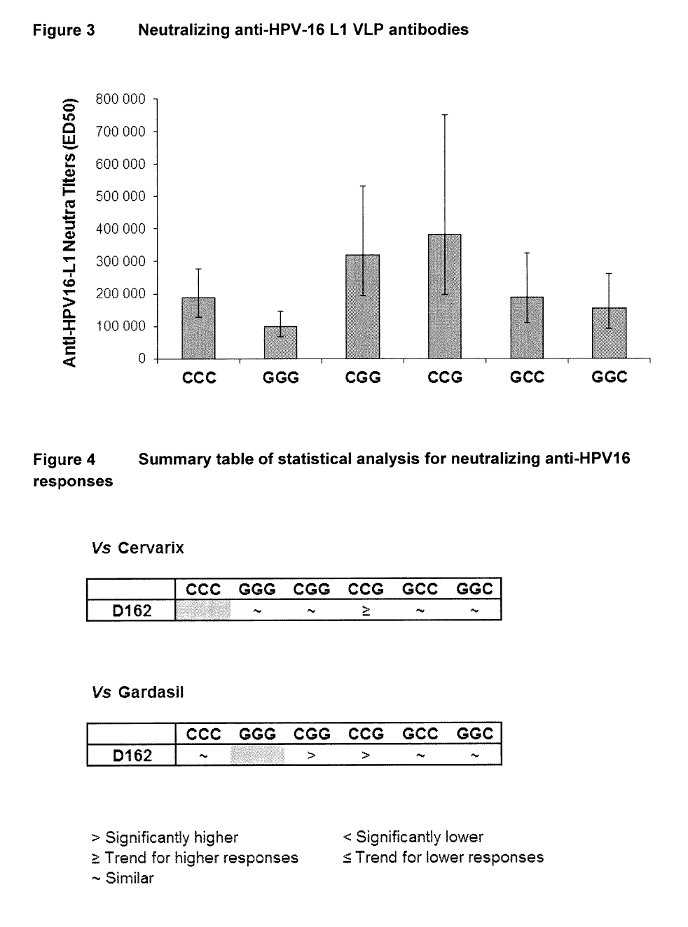

Comparison of neutralizing antibody responses (pseudo-neutralization assay—see below in Materials and Methods) following immunization with different vaccination schemes was performed at D42 post III and is presented in FIG. 3. Summary of statistical analysis comparing all groups to Cervarix™ or Gardasil™ control groups is presented in FIG. 4.

2. HPV-18 L1 VLP Responses

2.1 Total Antibody Response HPV18 (ELISA, Post II and III)

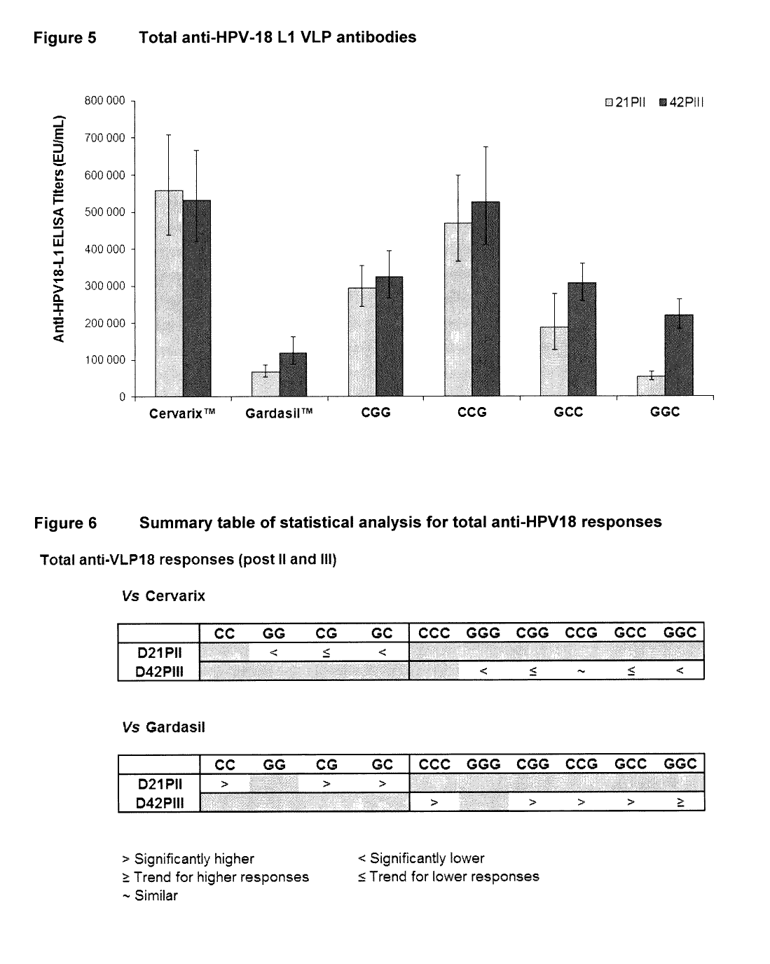

Comparison of total antibody responses (ELISA) following immunization with different vaccination schemes is presented in FIG. 5. Summary of statistical analysis comparing all groups to Cervarix™ or Gardasil™ control groups is presented in FIG. 6.

2.2 Neutralization Response HPV18 (PBNA, D42 PIII)

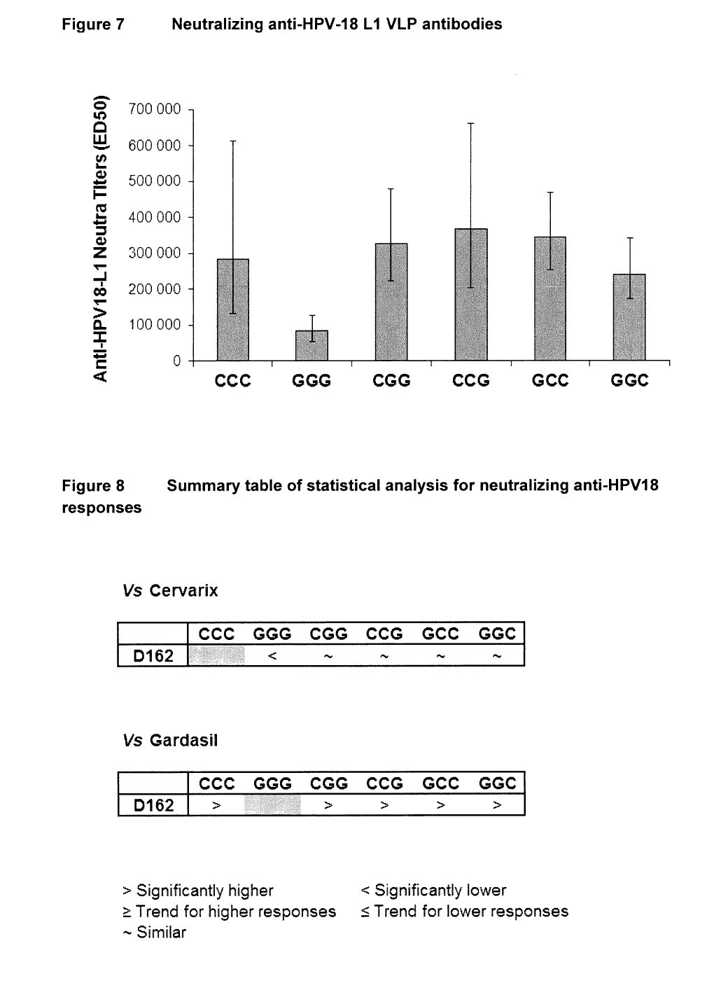

Comparison of neutralizing antibody responses (pseudo-neutralization assay) following immunization with different vaccination schemes was performed at D42 post III and is presented in FIG. 7. Summary of statistical analysis comparing all groups to Cervarix™ or Gardasil™ control groups is presented in FIG. 8.

3. HPV-6 L1 VLP Responses

3.1 Total Antibody Response HPV6 (ELISA, Post II and III)

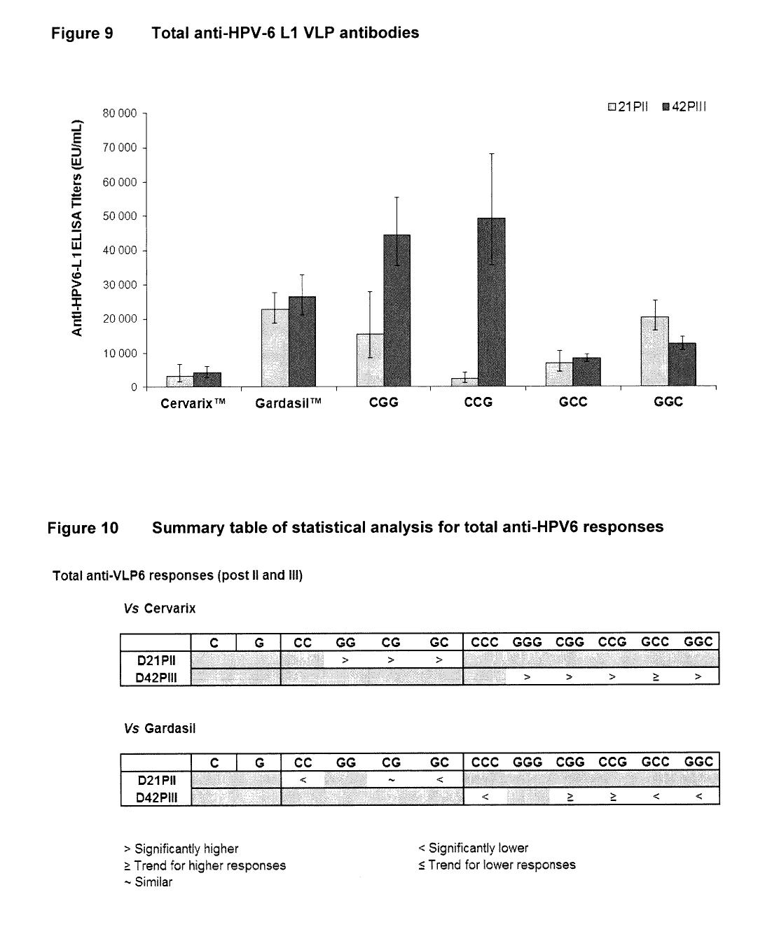

Comparison of total antibody responses (ELISA) following immunization with different vaccination schemes is presented in FIG. 9. Summary of statistical analysis comparing all groups to Cervarix™ or Gardasil™ control groups is presented in FIG. 10.

3.2 Neutralization Response HPV6 (PBNA, D42 PIII)

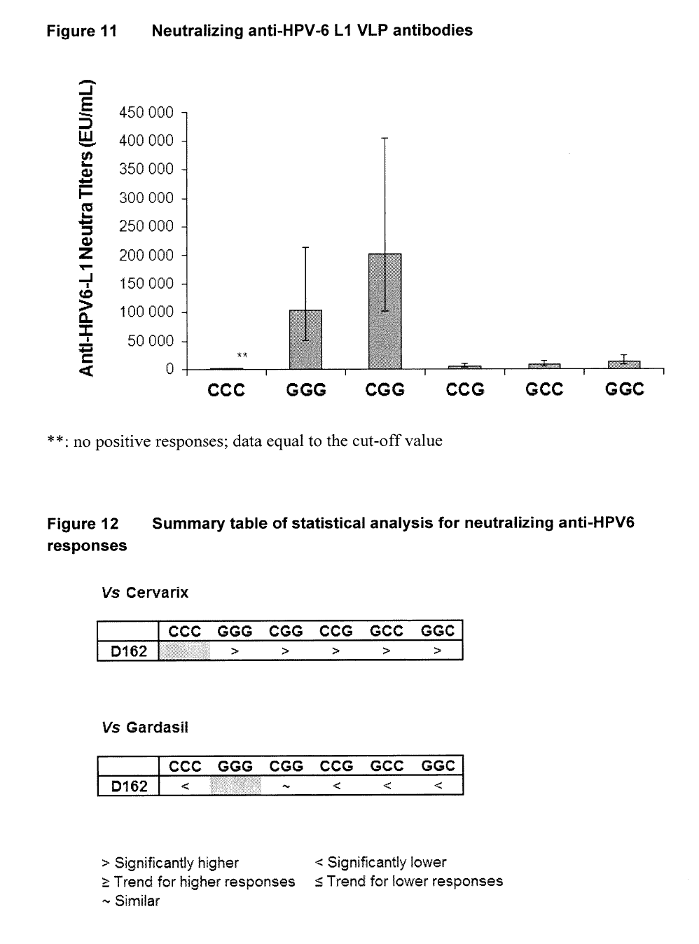

Comparison of neutralizing antibody responses (pseudo-neutralization assay) following immunization with different vaccination [symbol] performed at D42 post III and is presented in FIG. 11. Summary of statistical analysis comparing all groups to Cervarix™ or Gardasil™ control groups is presented in FIG. 12.

14. HPV-11 L1 VLP Responses

4.1 Total Antibody Response HPV11 (ELISA, Post II and III)

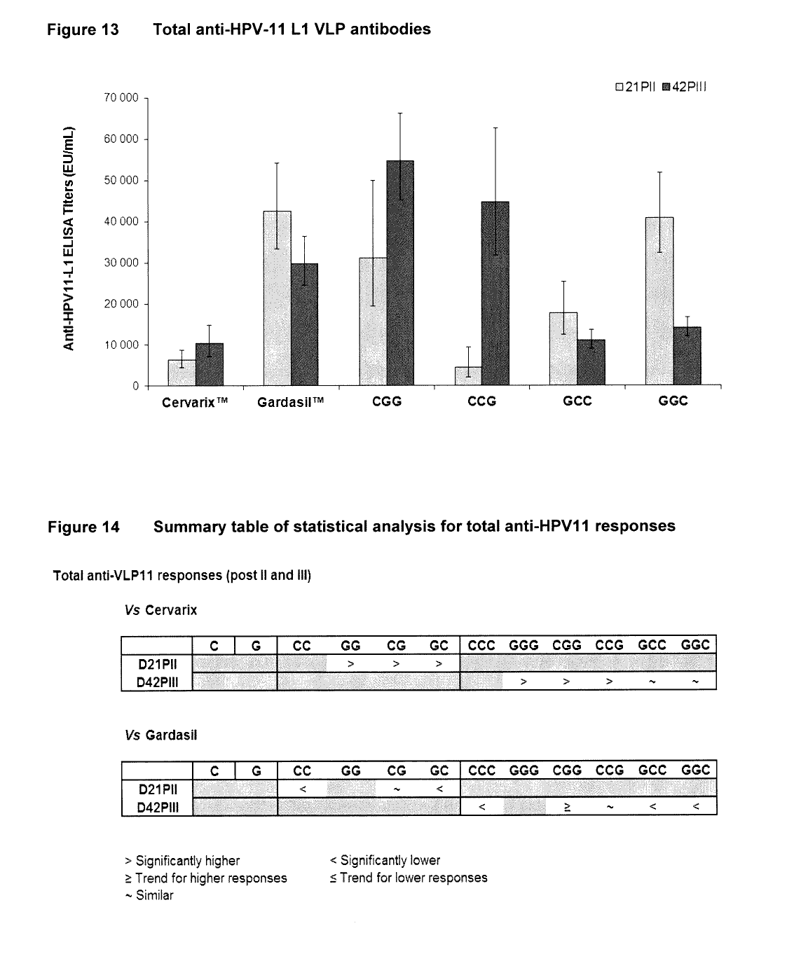

Comparison of total antibody responses (ELISA) following immunization with different vaccination schemes is presented in FIG. 13. Summary of statistical analysis comparing all groups to Cervarix™ or Gardasil™ control groups is presented in FIG. 14.

4.2 Neutralization Response HPV11 (PBNA, D42 PIII)

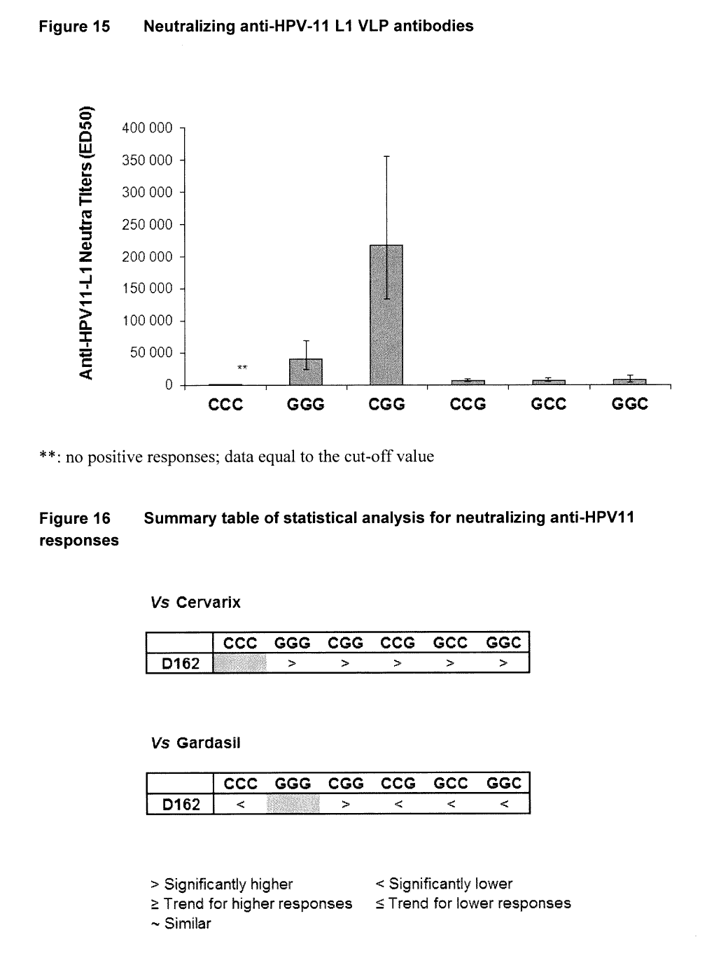

Comparison of neutralizing antibody responses (pseudo-neutralization assay) following immunization with different vaccination schemes was performed at D42 post III and is presented in FIG. 15. Summary of statistical analysis comparing all groups to Cervarix™ or Gardasil™ control groups is presented in FIG. 16.

Conclusions
Positive impact (3.5 to 32 fold, p<0.0001) of 1 dose Cervarix™ priming on total and neutralizing anti-HPV-6 and 11 responses in post III compared to Gardasil™ priming→CGG>GCC and GGC
Positive impact (3.1 to 5.8 fold, p<0.0001) of 2 dose Cervarix™ priming only on total anti-HPV-6 and 11 responses in post III compared to Gardasil™ priming→CCG≧GCC and GGC
Positive impact (1.9 to 2.6 fold, p≦0.0006) of Cervarix™ priming (1 or 2 doses) on total and neutralizing anti-HPV-16 responses at day 42 post III compared to Gardasil™ priming→CCG˜CGG≧GGG, GCC and GGC
Positive impact (1.7 to 4.2 fold, p≦0.0066) of 2 dose Cervarix™ priming on total anti-HPV-18 responses at day 42 post III compared to Gardasil™ priming→CCG≧GGG, GCC and GGC

A comparison between Cervarix™ Gardasil™ priming showed a reproducible positive impact of Cervarix™ priming on the ELISA antibody responses to all HPV L1 VLPs including 6 & 11 and PBNA responses to HPV-16, 6 and 11.

It was also shown that one priming with Cervarix™ was sufficient to induce antibody responses similar to Cervarix™ for HPV-16 but at least 2 doses of Cervarix™ priming were needed to ensure similar titers to Cervarix™ for HPV-18.

In conclusion, the immunogenicity data demonstrate the added value of priming with Cervarix™ at least 1× (HPV-16, 6 & 11) or 2× (HPV-18) compared to a complete vaccination schedule with Cervarix™ or Gardasil™.

TABLE
Vaccination schemes ranking for HPV 6 and HPV 11 based on total and
neutralizing antibody responses

| ELISA 6/11 | Vaccine | PBNA 6/11 |
|---|---|---|
| +++ | CGG | ++++ |
| ++ | GGG | +++ |
| ++ | GGC | + |
| + | GCC | +/− |
| +++ | CCG | +/− |
| + | CCC | − |

The added value of priming with 1 dose of Cervarix™ is maintained in a 2 dose scheme with 1/50th HD by demonstrating higher total anti-HPV18 responses and similar total anti-HPV11 responses compared to 2 doses of Gardasil™. See Example 2.

Materials and Methods

Anti-HPV 16/18/6/11 L1 VLPs ELISA

Quantification of anti-HPV-16/18/6/11 L1 VLPs antibodies was performed by ELISA using HPV-16, HPV-18, HPV-6 and HPV-11 truncated L1 VLPs as coating. Antigens were diluted at a final concentration of 1, 2 or 5 μg/ml in PBS and were adsorbed overnight at 4° C. to the wells of 96-wells microtiter plates (Maxisorp Immuno-plate, Nunc, Denmark). The plates were then incubated for 1 hr at 37° C. with PBS containing 0.1% Tween20+1% BSA (saturation buffer). Sera diluted in saturation buffer were added to the HPV L1-coated plates and incubated for 1 hr 30 min at 37° C. The plates were washed four times with PBS 0.1% Tween20 and biotin-conjugated anti-mouse Ig (Dako, UK) diluted in saturation buffer was added to each well and incubated for 1 hr 30 at 37° C. After a washing step, streptavidin-horseradish peroxydase (Dako, UK), diluted in saturation buffer was added for an additional 30 min at 37° C. Plates were washed as indicated above and incubated for 20 min at room temperature with a solution of 0.04% o-phenylenediamine (Sigma) 0.03% H₂O₂ in 0.1% Tween20, 0.05M citrate buffer pH 4.5. The reaction was stopped with 2N H2SO4 and read at 492/620 nm. ELISA titers were calculated from a reference by SoftMaxPro (using a four parameters equation) and expressed in EU/ml.

Pseudo-Neutralization Assay (PBNA)

Pseudoviruses (PsV) were generated by transfection of 293TT cells (human embryonic kidney cell line+SV40 T antigen) with both L1 and L2 expressing plasmids and reporter plasmid p2CMVSEAP (SEAP=secreted alkaline phosphatase). Briefly, 20 million 293TT cells were plated 16 h before transfection. For example: for the production of HPV16 pseudovirus, the cells were transfected (Lipofectamine 2000/Invitrogen) with 27 μg each of pYSEAP, p16L1h, and p16L2h and then harvested 40-48 post-transfection. The extracted pseudo-virion particles were than further purified using Optiprep (Sigma). Preparations were inspected for purity on 10% SDS-Tris-glycine gels (Bio-Rad), titrated on 293 TT cells to test for infectivity by SEAP detection (Chemiluminescence, BD Clontech), then pooled and frozen at −80 ° C. until use.

To assay the neutralizing titers in serum samples, 293TT target cells were pre-plated 3-4 h in advance in 96-well flat bottom plates at 30,000 cells/well. Pseudovirus preparations were diluted appropriately to obtain alkaline phosphatase (SEAP) for an output reading of 30-70 relative light units (RLUs). Diluted pseudovirus stocks were placed in 96-well plates and combined with diluted serum, and placed on ice for 1 h. The pseudovirus-antibody mixture were then transferred onto the pre-plated cells and incubated for 72 h. At the end of the incubation, the supernatant was harvested and clarified at 1500×g for 5 min. The SEAP content in the clarified supernatant was determined using the Great ESCAPE SEAP Chemiluminescence Kit (BD Clontech) as directed by the manufacturer. Twenty minutes after the substrate was added, samples were read in either white Microlite 1 (Dynex) or Optiplate-96 (Perkin-Elmer) opaque 96-well plates for 0.20 s/well using an MLX Microplate Luminometer (Dynex Technologies) set at Glow-Endpoint.

Serum neutralization titers were defined as the reciprocal of the highest dilution that caused at least a 50% reduction in SEAP activity compared to the control without serum. A serum was considered to be positive for neutralization in the HPV-16, HPV-18, HPV-6 and HPV-11 assay if it was neutralizing at a dilution at least 4-fold higher than the titer observed in the BPV1 neutralization assay (negative control).

Statistical Analysis

The group means were compared using a one-way analysis of variance (ANOVA 1). The analysis was conducted on log 10 transformed data for normalization purpose. When a significant difference between group means was detected (pvalue<0.05), pairwise comparisons among means were performed at a 0.05 significant level (Tukey-HSD comparison test).

UL/LL=Upper/lower limits of the 95% confidence interval (CI).

### Example 2

### Two Dose Immunisation Study in Mice Including Challenge Study

This preclinical experiment was launched in order to compare the specific protection induced against HPV-18 and 11 after vaccination with CC, CG, GG or GC schemes. In this experiment the vaccines were used at a dose of 1/50^{th} of the human dose.

Part I—Immunogenicity Study

BALB/c mice (10 mice per group) received intramuscular injections at days 0 and 21 days with 2 doses of Cervarix™ 1/50 HD, 2 doses of Gardasil™ 1/50 HD, 1 dose of Cervarix™ 1/50 HD followed by 1 dose of Gardasil™ 1/50 HD or 1 dose of Gardasil™ 1/50 HD followed by 1 dose of Cervarix™ 1/50 HD.

Blood was collected at day 28 post II and analysed by ELISA for total antibody titers against HPV-18 and 11 L1 VLPs after vaccination with CC, GG, CG or GC. Neutralizing antibody titers against HPV-18 and 11 were also measured (by PBNA) at day 28 post II.

Mice were challenged with PsV-18 and 11 at 1 month post II to evaluate specific and cross-protection induced with those different immunisation schemes.

Groups

| Groups | D0 | D21 | Challenge M1 PII | |
|---|---|---|---|---|
| 1 | Cervarix ™ | Cervarix ™ | Luc. PsV18 | Luc. PsV11 |
| | | | (n = 5) | (n = 5) |
| 2 | Gardasil ™ | Gardasil ™ | Luc. PsV18 | Luc. PsV11 |
| | | | (n = 5) | (n = 5) |
| 3 | Cervarix ™ | Gardasil ™ | Luc. PsV18 | Luc. PsV11 |
| | | | (n = 5) | (n = 5) |
| 4 | Gardasil ™ | Cervarix ™ | Luc. PsV18 | Luc. PsV11 |
| | | | (n = 5) | (n = 5) |
| 5 | NaCl | NaCl | Luc. PsV18 | Luc. PsV11 |
| | | | (n = 5) | (n = 5) |

Luc. PsV18 = HPV 18 pseudovirus containing luciferase reporter gene

Adjuvant Formulations ( 1/50 HD)

| Formulations | Aluminium | MPL |
|---|---|---|
| Cervarix ™ | 10 μg Al(OH)₃ | 1 μg |
| Gardasil ™ | 4.5 μg MAA* | — |

*MAA = Merck Aluminium hydroxyphosphate sulfate

Results

Humoral responses to HPV-18 and 11 L1 VLPs after injection of different immunization schemes were monitored by the total (ELISA) antibody and neutralizing (PBNA) antibody responses.

1. HPV-18 L1 VLP Responses

1.1 Total Antibody Response HPV-18 (ELISA, D28 PII)

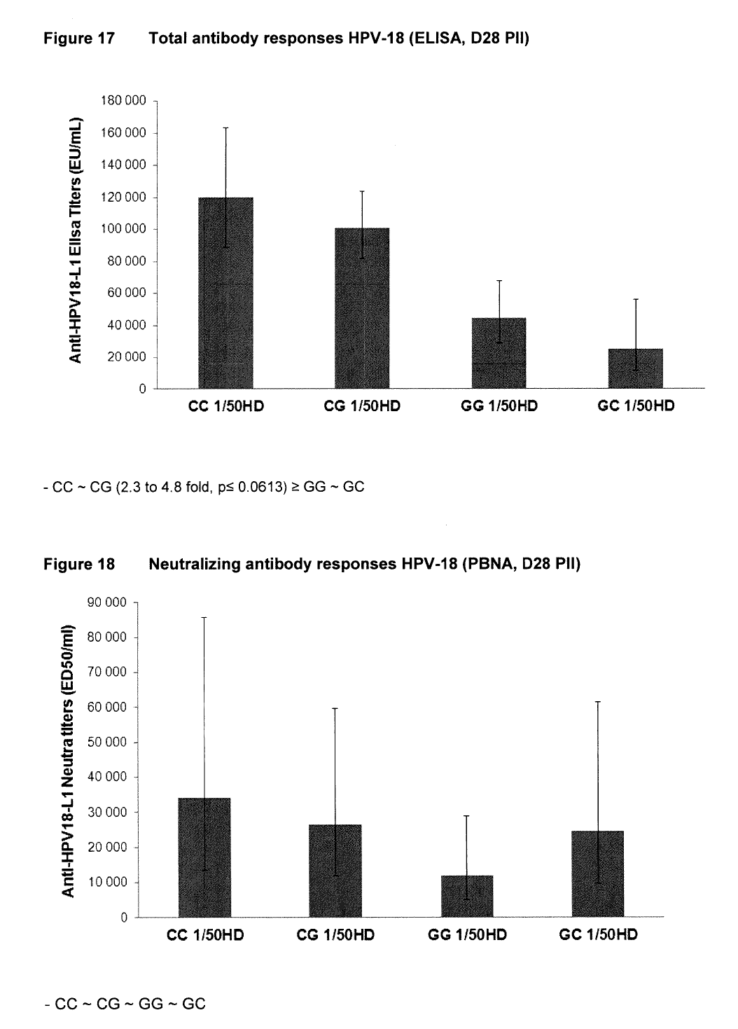

Comparison of total antibody responses (ELISA) at Day 28 PII following immunization with different vaccination schemes is presented in FIG. 17.
CC˜CG (2.3 to 4.8 fold, p≦0.0613)≧GG˜GC

1.2 Neutralizing Antibody Response HPV-18 (PBNA, D28 PII)

Comparison of neutralizing antibody responses (pseudo-neutralization assay—see Materials and Methods for Example 1) following immunization with different vaccination schemes is presented in FIG. 18.
CC˜CG˜GG˜GC

2. HPV-11 L1 VLP Responses

2.1 Total Antibody Response HPV-11 (ELISA, D28 PII)

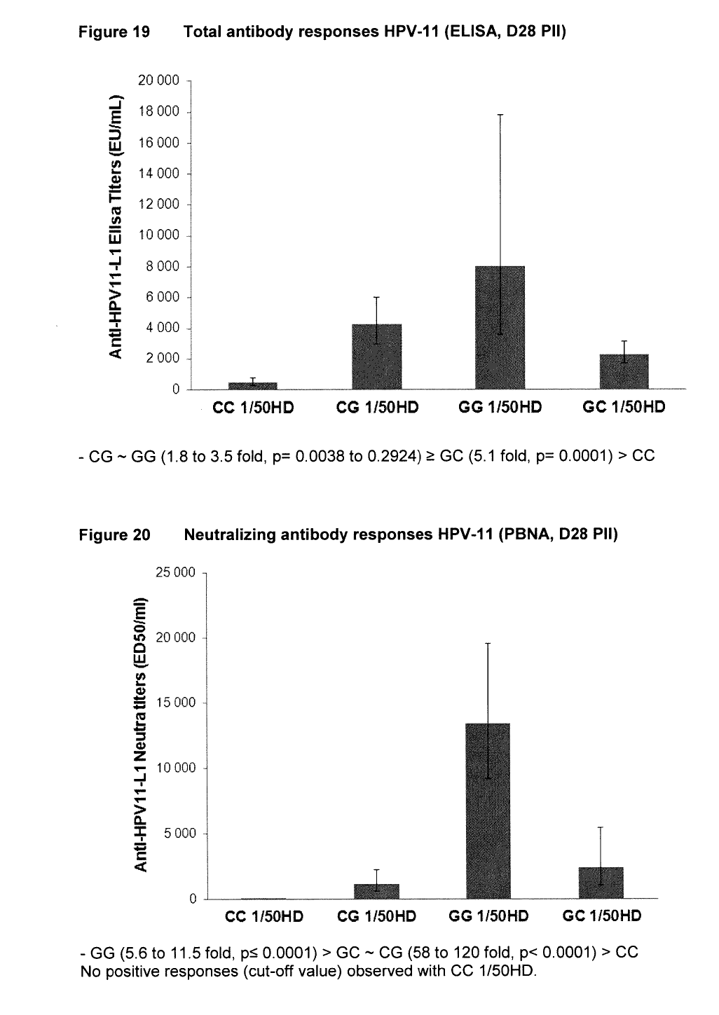

Comparison of total antibody responses (ELISA) following immunization with different vaccination schemes is presented in FIG. 19.
CG˜GG (1.8 to 3.5 fold, p=0.0038 to 0.2924)≧GC (5.1 fold, p=0.0001)>CC

2.2 Neutralizing Antibody Response HPV-11 (PBNA, D28 PII)

Comparison of neutralizing antibody responses (pseudo-neutralization assay NCI) following immunization with different vaccination schemes is presented in FIG. 20.
GG (5.6 to 11.5 fold, p≦0.0001)>GC˜CG (58 to 120 fold, p<0.0001)>CC
No positive responses (cut-off value) observed with CC 1/50 HD.

Conclusions

Similar total anti-VLP18 titers were observed with CC and CG and these were higher than GG and GC schemes.

There was a statistically significant higher total anti-VLP11 response with GG and CG compared to GC and CC.

There were similar specific neutralizing antibody titers to HPV-18 with all vaccination schemes.

There was statistically significant lower neutralizing antibody titers to HPV-11 with CG compared to GG but higher than CC scheme.

Part II—Intravaginal Challenge and Protection

Specific protection induced after CC, GG, CG or GC vaccination schemes was evaluated 1 month post II following challenge of vaccinated mice with Luciferase PsV-18 and 11 (see Materials and Methods below).

1. PsV-18 Challenge

Following unexpected protection (60%) observed with the NaCl group (i.e. non-vaccine control) it was not possible to conclude on protection levels after challenge with PsV-18 (data therefore not presented).

2. PsV-11 Challenge

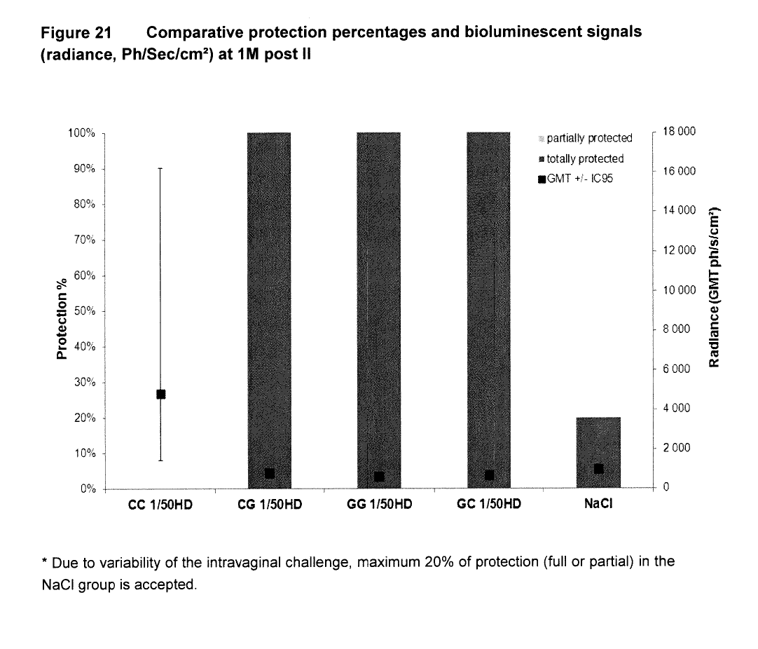

Comparison of protection against PsV-11 induced after CC, GG, CG or GC vaccination is presented in FIG. 21.

Note: Due to variability of the intravaginal challenge, maximum 20% of protection (full or partial) in the NaCl group is accepted.

| | CC | CG | GG | GC | |
|---|---|---|---|---|---|
| | 1/50HD | 1/50HD | 1/50HD | 1/50HD | NaCl |
| Protection % | 0% | 100% | 100% | 100% | 20% |
| (M1 PII) | | | | | |
| ELISA titers (EU/ml) | 452 | 4250 | 8003 | 2312 | 35 |
| PBNA titers | 20 | 1168 | 13429 | 2392 | NT |
| (ED50/ml) | | | | | |

Full protection (100%) percentages observed with GG, CG and GC
No protection with CC vaccination
No protection observed when no neutralizing antibody responses measured

Conclusions

Despite lower neutralizing responses to HPV-11 with CG and GC vaccination schemes compared to GG, 100% protection against PsV-11 was observed with those 2 schemes. Moreover, absence of neutralizing antibodies to HPV-11 was observed with CC vaccination in parallel with no protection to this same type suggesting a correlation between presence of neutralizing antibodies and protection percentage.

Result Highlights:
Total antibodies (ELISA)
   HPV-18: CC˜CG≧GG˜GC
   HPV-11: GG˜CG≧GC>CC
Neutralizing antibodies (PBNA: HPV-6/11)
   HPV-18: CC˜CG˜GG˜GC
   HPV-11: GG>GC˜CG>CC
Efficacy (intravaginal challenge mice model)
   HPV-18: data inconclusive following unexpected protection in the NaCl groups
   HPV-11: GG˜CG˜GC>CC vaccination with 100% full protection vs 0%

Conclusions

Priming with 1 dose of Cervarix™ followed by 1 dose of Gardasil™ induced similar total anti-HPV-18 responses to two doses of Cervarix™ (CC) and similar anti-HPV-11 responses compared to 2 doses of Gardasil™ (GG). Moreover, 100% protection was observed to PsV-11 with CG vaccination as for GG. These observations confirm the potential added value to begin vaccination scheme with Cervarix™ based on ELISA titers and protection percentages.

Materials and Methods

In Vivo Challenge

Two weeks post immunization, mice were subcutaneously injected with 3 mg/100 μl of Depo-Provera to synchronize the hormonal cycle of the mice. Four days later, mice were intravaginally pre-treated with 50 μl Conceptrol, a CMC-based spermicide containing 4% Nonoxynol-9 used to disrupt the epithelium of vaginal tract. The mice were intravaginally challenged six hours later with 30 μl Luciferase-Pseudovirions diluted in 1.5% Low viscosity Carboxymethylcellulose. The pseudovirions are composed of HPV L1 and L2 surface proteins that have encapsulated reporter plasmid expressing luciferase protein. PsV infection was monitored by measuring luciferase expression in the genital tract on day 2 post challenge. Anesthetized mice were instilled intravaginally with 20 μl luciferin (15 mg/ml) and imaged 5 minutes later during 2 minutes exposure using a Xenogen IVIS Spectrum in vivo imager (Caliper LifeSciences).

Protection was defined if mice had a signal inferior to average+3 SD of the signal obtained with NaCl vaccinated mice challenged with a PsV-18 expressing SEAP (negative control).

Mice were considered as fully protected when bioluminescent signal obtained after challenge was below the cut-off value of 939 ph/sec/cm². This value was determined by statisticians using bioluminescent signals measured in the irrelevant thoracic zone (#10 experiments). Mice were considered as partially protected when bioluminescent signal measured was higher than the cut-off value of 939 ph/sec/cm² but below the lower limit of the C195 observed for the negative NaCl control group.

### Example 3

### Comparative Short and Long Term Protection Induced with Cervarix™ and Gardasil™ Vaccines in a 3 Dose Vaccination Scheme (Day 0, 45, 120, at 1/50^{th} of the Human Dose)

These preclinical experiments were launched in order to compare the specific and cross protection induced against HPV-18/6 and 11 after vaccination with CCC, GGG, CGG, CCG, GCC and GGC schemes. This was evaluated at 1 and 6 months post III to mimic short and long term protection. The vaccination scheme D0/45/120 was used to mimic a 0/M2/M6 vaccination scheme in the clinics.

BALB/c mice (20 mice per group) received intramuscular injections at days 0, 45 and 120. Two groups received 3 injections of Cervarix™ 1/50th HD (HPV-16/18 L1 VLPs 0.4/0.4 μg+AS04) or Gardasil™ 1/50th HD (HPV-16/18/6/11 L1 VLPs 0.8/0.4/0.4/0.8 μg+MAA*) vaccines. Four other additional groups were injected with Cervarix™ 1/50th HD at day 0 and Gardasil™ 1/50th HD at days 45 and 120; Cervarix™ 1/50th HD at days 0 and 45 followed by Gardasil™ 1/50th HD at day 120; Gardasil™ 1/50th HD at day 0 followed by Cervarix™ 1/50th HD at days 45 and 120 or Gardasil™ 1/50th HD at days 0 and 45 followed by Cervarix™ 1/50th HD at day 120.

Blood was collected at 1 month post III (20100801) or 6 months post III (20100810) and just before challenge to analyse total antibody titers (ELISA) against HPV-18/6 and 11 L1 VLPs. Neutralizing antibody titers (PBNA) against HPV-18/6 and 11 were also measured at 1 or 6 months post III.

Mice were challenged with PsV-18/6 or 11 at 1 month or 6 months after the third dose to evaluate specific protection induced with these different immunisation schemes.

Groups

| Groups | D0 | D45 | D120 |
|---|---|---|---|
| 1 | Cervarix ™ | Cervarix ™ | Cervarix ™ |
| 2 | Gardasil ™ | Gardasil ™ | Gardasil ™ |
| 3 | Cervarix ™ | Gardasil ™ | Gardasil ™ |
| 4 | Cervarix ™ | Cervarix ™ | Gardasil ™ |
| 5 | Gardasil ™ | Cervarix ™ | Cervarix ™ |
| 6 | Gardasil ™ | Gardasil ™ | Cervarix ™ |

Adjuvant Formulations ( 1/50 Human Dose)

| Formulations | Aluminium | MPL |
|---|---|---|
| Cervarix ™ AHPVA044A | 10 μg Al(OH)₃ | 1 μg |
| Gardasil ™ NJ17990 | 4.5 μg MAA* | — |

*MAA = Merck Aluminium hydroxyphosphate sulfate

Results

Humoral responses to HPV-18, 6 and 11 L1 VLPs after injection of different immunization schemes were monitored by the total (ELISA) antibody and neutralizing (PBNA) antibody responses at 1 month or 6 months post vaccination.

1.1. Humoral Responses

1.1.1. HPV-18 L1 VLP Responses

1.1.1.1. Total Antibody Response HPV-18 (ELISA, 1 or 6M PIII)

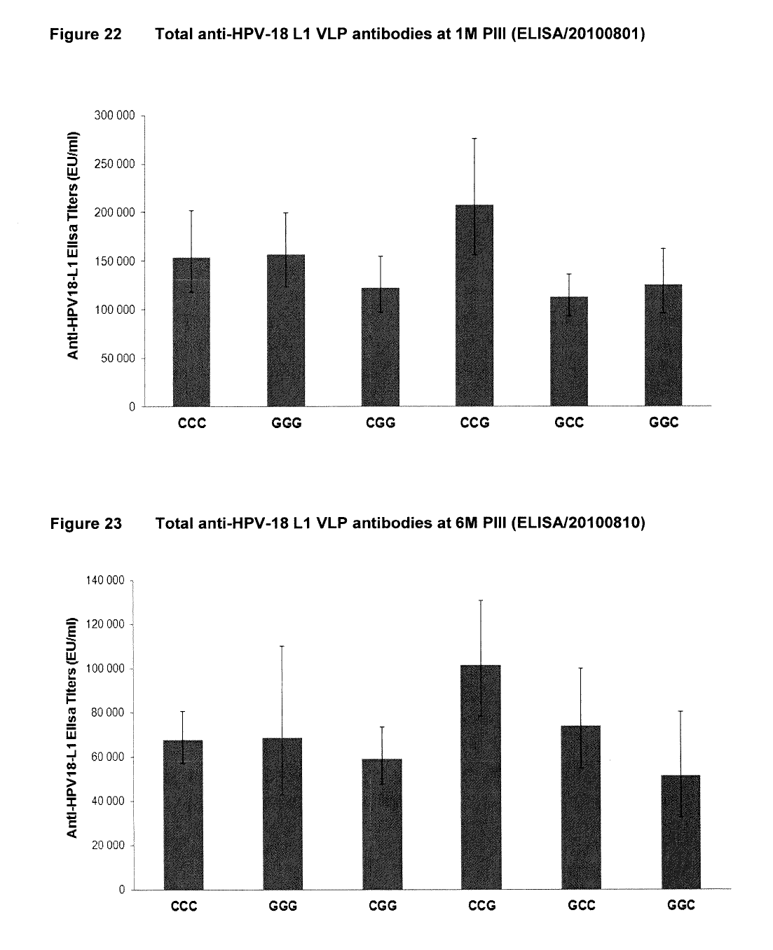

Comparisons of total antibody responses (ELISA) at 1M and 6M PIII following immunization with different vaccination schemes are presented in FIGS. 22 and 23.

Summaries of statistical analyses are as follows:

Vs Cervarix

| | CCC | GGG | CGG | CCG | GCC | GGC |
|---|---|---|---|---|---|---|
| D30 PIII | | ~ | ~ | ~ | ~ | ~ |

Vs Gardasil

| | CCC | GGG | CGG | CCG | GCC | GGC |
|---|---|---|---|---|---|---|
| D30 PIII | ~ | | ~ | ~ | ~ | ~ |

Vs Cervarix

| | CCC | GGG | CGG | CCG | GCC | GGC |
|---|---|---|---|---|---|---|
| 6M PIII | | ~ | ~ | ~ | ~ | ~ |

Vs Gardasil

| | CCC | GGG | CGG | CCG | GCC | GGC |
|---|---|---|---|---|---|---|
| 6M PIII | ~ | | ~ | ~ | ~ | ~ |

1.1.1.2. Neutralizing Antibody Response HPV-18 (ELISA, 1 or 6M PIII)

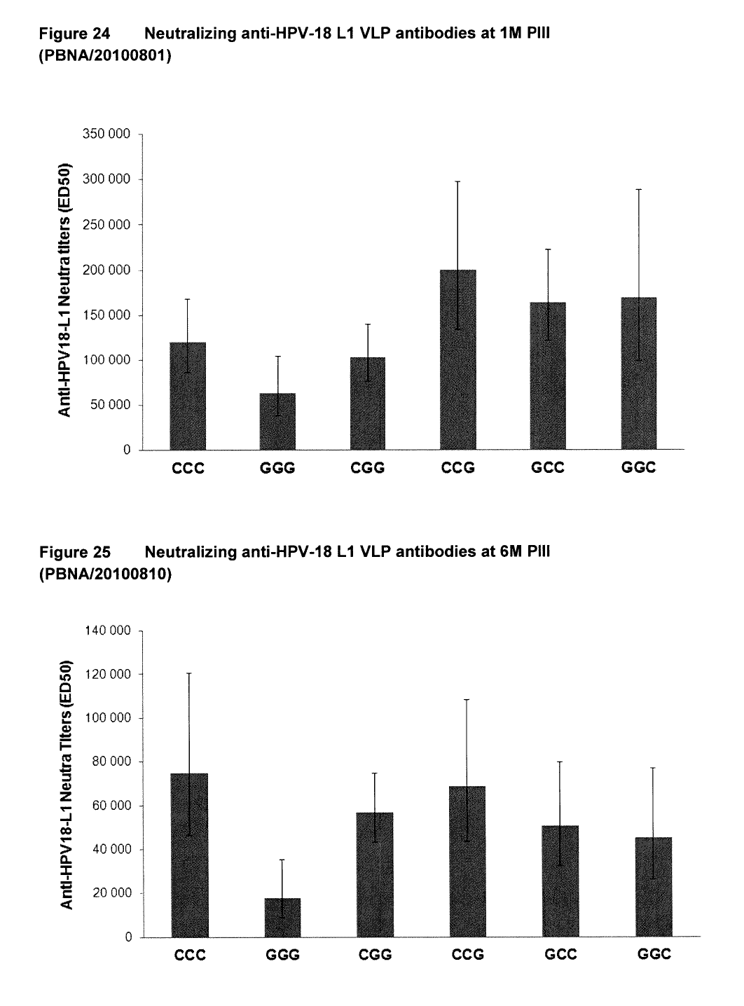

Comparisons of neutralizing antibody responses (ELISA) at 1M and 6M PIII following immunization with different vaccination schemes are presented in FIGS. 24 and 25.

Summaries of statistical analyses are as follows:

Vs Cervarix

| | CCC | GGG | CGG | CCG | GCC | GGC |
|---|---|---|---|---|---|---|
| D30 PIII | | ~ | ~ | ~ | ~ | ~ |

Vs Gardasil

| | CCC | GGG | CGG | CCG | GCC | GGC |
|---|---|---|---|---|---|---|
| D30 PIII | ~ | | ~ | > | > | > |

Vs Cervarix

| | CCC | GGG | CGG | CCG | GCC | GGC |
|---|---|---|---|---|---|---|
| 6M PIII | | < | ~ | ~ | ~ | ~ |

Vs Gardasil

| | CCC | GGG | CGG | CCG | GCC | GGC |
|---|---|---|---|---|---|---|
| 6M PIII | > | | > | > | > | ~ |

1.1.2. HPV-6 L1 VLP Responses

1.1.2.1. Total Antibody Response HPV-6 (ELISA, 1 or 6M PIII)

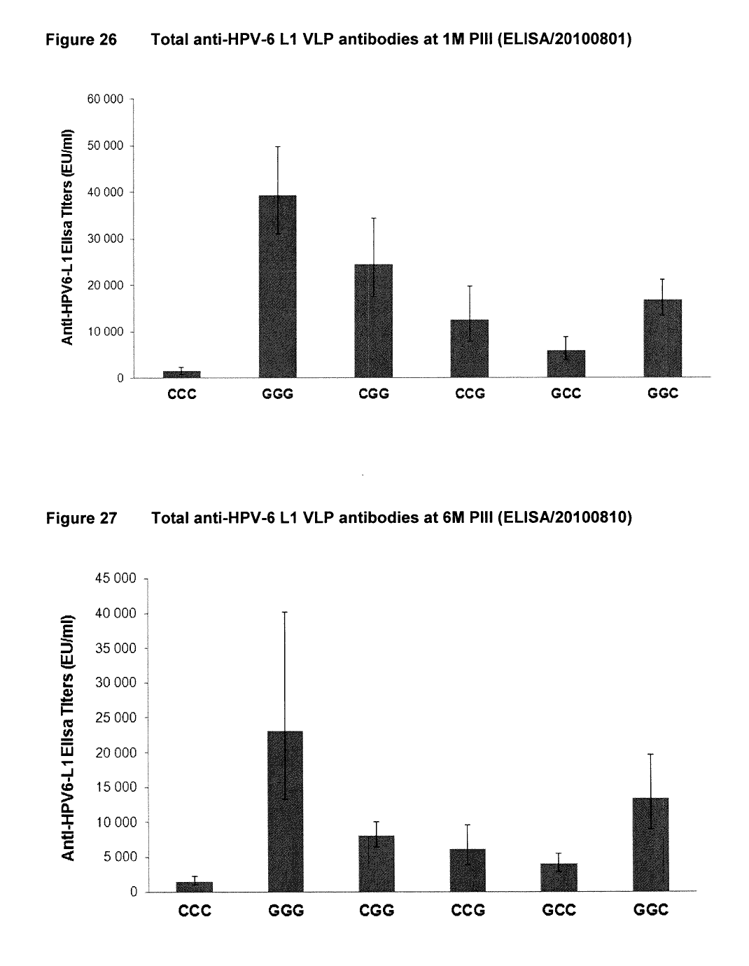

Comparisons of total antibody responses (ELISA) at 1M and 6M PIII following immunization with different vaccination schemes are respectively presented in FIGS. 26 and 27.

Summaries of statistical analyses are as follows:

Vs Cervarix

| | CCC | GGG | CGG | CCG | GCC | GGC |
|---|---|---|---|---|---|---|
| D30 PIII | | > | > | > | > | > |

Vs Gardasil

| | CCC | GGG | CGG | CCG | GCC | GGC |
|---|---|---|---|---|---|---|
| D30 PIII | < | | ~ | < | < | < |

Vs Cervarix

| | CCC | GGG | CGG | CCG | GCC | GGC |
|---|---|---|---|---|---|---|
| M6 PIII | | > | > | > | > | > |

Vs Gardasil

| | CCC | GGG | CGG | CCG | GCC | GGC |
|---|---|---|---|---|---|---|
| M6 PIII | < | | < | < | < | ~ |

1.1.2.2. Neutralizing Antibody Response HPV-6 (ELISA, 1 or 6M PIII)

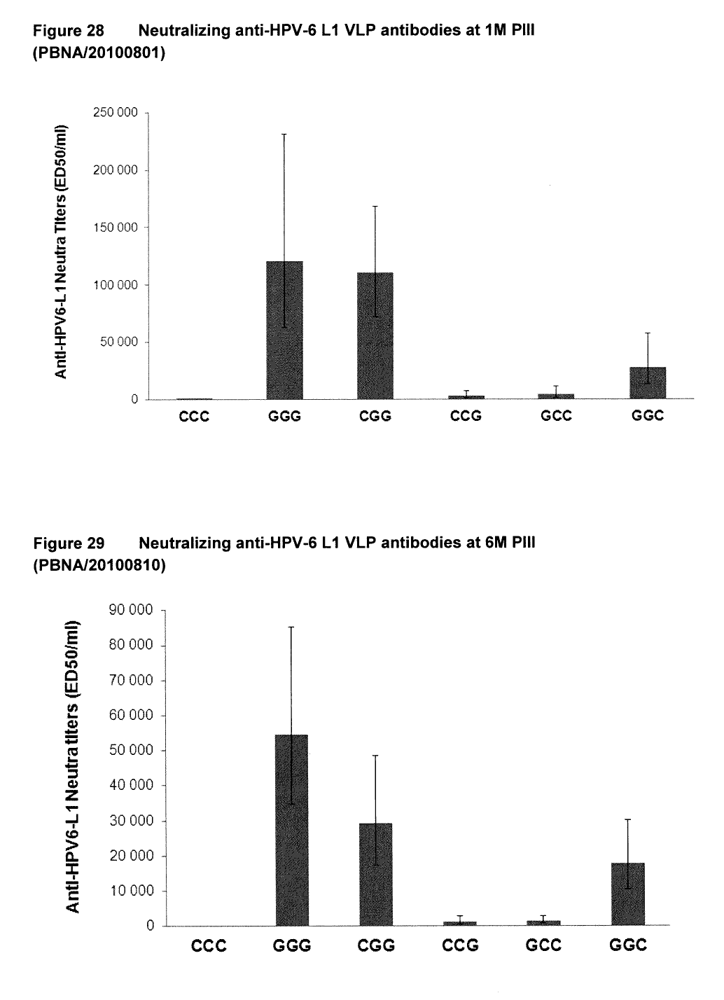

Comparisons of neutralizing antibody responses (ELISA) at 1M and 6M PIII following immunization with different vaccination schemes are respectively presented in FIGS. 28 and 29.

Summaries of statistical analyses are as follows:

Vs Cervarix

| | CCC | GGG | CGG | CCG | GCC | GGC |
|---|---|---|---|---|---|---|
| D30 PIII | | > | > | > | > | > |

Vs Gardasil

| | CCC | GGG | CGG | CCG | GCC | GGC |
|---|---|---|---|---|---|---|
| D30 PIII | < | | ~ | < | < | < |

Vs Cervarix

| | CCC | GGG | CGG | CCG | GCC | GGC |
|---|---|---|---|---|---|---|
| M6 PIII | | > | > | > | > | > |

Vs Gardasil

| | CCC | GGG | CGG | CCG | GCC | GGC |
|---|---|---|---|---|---|---|
| M6 PIII | < | | ~ | < | < | < |

1.1.3. HPV-11 L1 VLP Responses

1.1.3.1. Total Antibody Response HPV-11 (ELISA, 1 or 6M PIII)

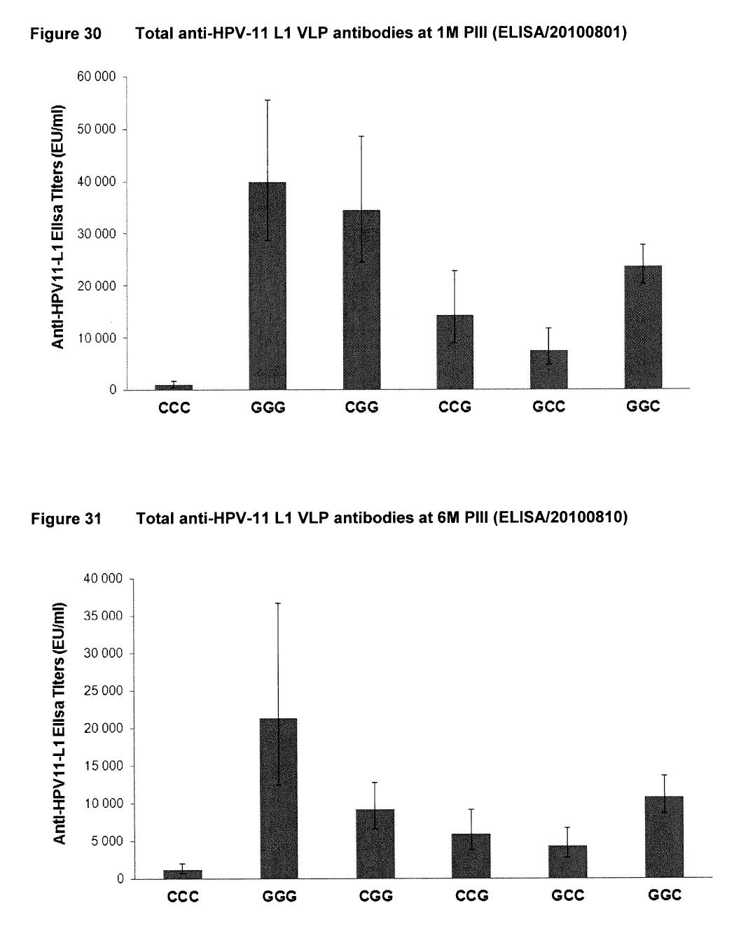

Comparisons of total antibody responses (ELISA) at 1M and 6M PIII following immunization with different vaccination schemes are presented in FIGS. 30 and 31.

Summaries of statistical analyses are as follows:

Vs Cervarix

| | CCC | GGG | CGG | CCG | GCC | GGC |
|---|---|---|---|---|---|---|
| D30 PIII | | > | > | > | > | > |

Vs Gardasil

| | CCC | GGG | CGG | CCG | GCC | GGC |
|---|---|---|---|---|---|---|
| D30 PIII | < | | ~ | < | < | ~ |

Vs Cervarix

| | CCC | GGG | CGG | CCG | GCC | GGC |
|---|---|---|---|---|---|---|
| M6 PIII | | > | > | > | > | > |

Vs Gardasil

| | CCC | GGG | CGG | CCG | GCC | GGC |
|---|---|---|---|---|---|---|
| M6 PIII | < | | < | < | < | ≦ |

1.1.3.2. Neutralizing Antibody Response HPV-11 (ELISA, 1 or 6M PIII)

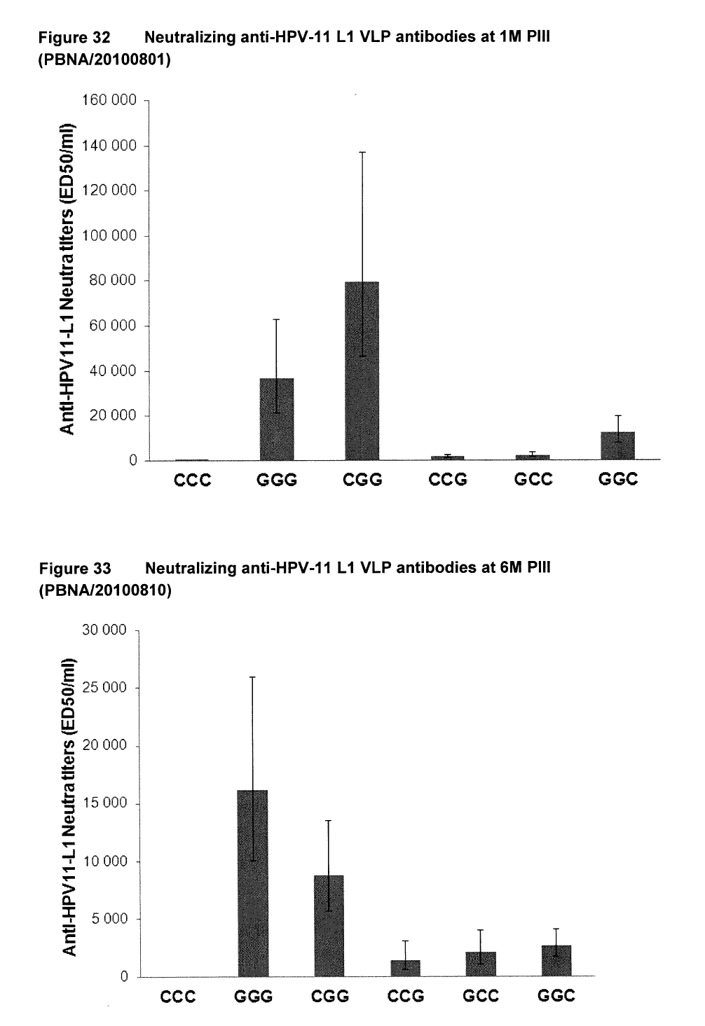

Comparisons of neutralizing antibody responses (ELISA) at 1M and 6M PIII following immunization with different vaccination schemes are respectively presented in FIGS. 32 and 33.

Summaries of statistical analyses are as follows:

Vs Cervarix

| | CCC | GGG | CGG | CCG | GCC | GGC |
|---|---|---|---|---|---|---|
| D30 PIII | | > | > | > | > | > |

Vs Gardasil

| | CCC | GGG | CGG | CCG | GCC | GGC |
|---|---|---|---|---|---|---|
| D30 PIII | < | | ~ | < | < | < |

Vs Cervarix

| | CCC | GGG | CGG | CCG | GCC | GGC |
|---|---|---|---|---|---|---|
| M6 PIII | | > | > | > | > | > |

Vs Gardasil

| | CCC | GGG | CGG | CCG | GCC | GGC |
|---|---|---|---|---|---|---|
| M6 PIII | < | | ~ | < | < | < |

1.1.4. Conclusions

There were similar (<2 fold, p=0.0051 to 1.000) total anti-HPV18 responses with all tested vaccination schemes 1 and 6 months post vaccination. Positive impact of Cervarix™ boost compared to classical Gardasil™ 3 doses and positive impact of 2× Cervarix™ priming on total anti-HPV18 responses compared to Gardasil™ priming not confirmed in this experiment.

This experiment did not show reproducible added value of 1× Cervarix™ priming on total and neutralizing anti-HPV-6 and 11 responses at 1 and 6 months post III compared to Gardasil™ priming.

See overall conclusions.

1.2. Intravaginal Challenge and Protection

Specific protection induced after different vaccination schemes was evaluated 1 month or 6 months post III following challenge of vaccinated mice with Luciferase PsV-18/6 or 11.

1.2.1. PsV-18 Challenge

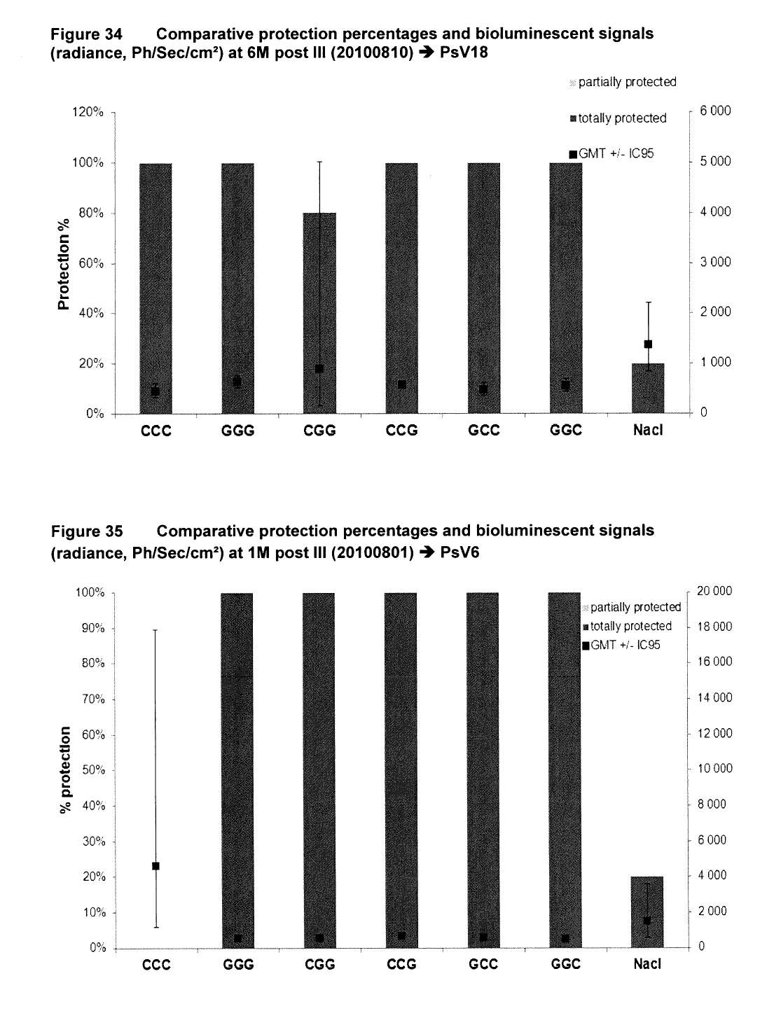

Comparison of protection percentages at 6M PIII following immunization with different vaccination schemes is presented in FIG. 34.

Following an unexpected finding of protection (80%) observed with the NaCl group 1 month after vaccination it was not possible to conclude on short-term protection levels after challenge with PsV-18 (data not presented).
Due to variability of the intravaginal challenge, maximum 20% of protection (full or partial) in the NaCl group is accepted.

| | CCC | GGG | CGG | CCG | GCC | GGC | NaCl |
|---|---|---|---|---|---|---|---|
| Protection % (M6 PIII) | 100% | 100% | 80% | 100% | 100% | 100% | 20% |

100% protection was observed with all vaccination schemes except with CGG (80%)

1.2.2. PsV-6 Challenge

Comparison of protection percentages at 1M and 6M PIII following immunization with different vaccination schemes is presented in FIGS. 35 and 36 respectively.
Due to variability of the intravaginal challenge, maximum 20% of protection (full or partial) in the NaCl group is accepted.

| | CCC | GGG | CGG | CCG | GCC | GGC | NaCl |
|---|---|---|---|---|---|---|---|
| Protection % (M1 PIII) | 0% | 100% | 100% | 100% | 100% | 100% | 20% |

At 1 month post III 100% protection was observed with GGG, CGG, CCG, GCC and GGC by contrast to CCC vaccination which was without any protection against PsV-6→GGG˜CGG˜CCG˜GCC˜GGC>CCC

| | CCC | GGG | CGG | CCG | GCC | GGC | NaCl |
|---|---|---|---|---|---|---|---|
| Protection % (M6 PIII) | 0% | 100% | 100% | 100% | 100% | 100% | 0% |

At 6 months post III 100% protection was observed with GGG, CGG, CCG, GCC and GGC by contrast to CCC vaccination which was without any protection against PsV-6→GGG˜CGG˜CCG˜GCC˜GGC>CCC

1.2.3. PsV-11 Challenge

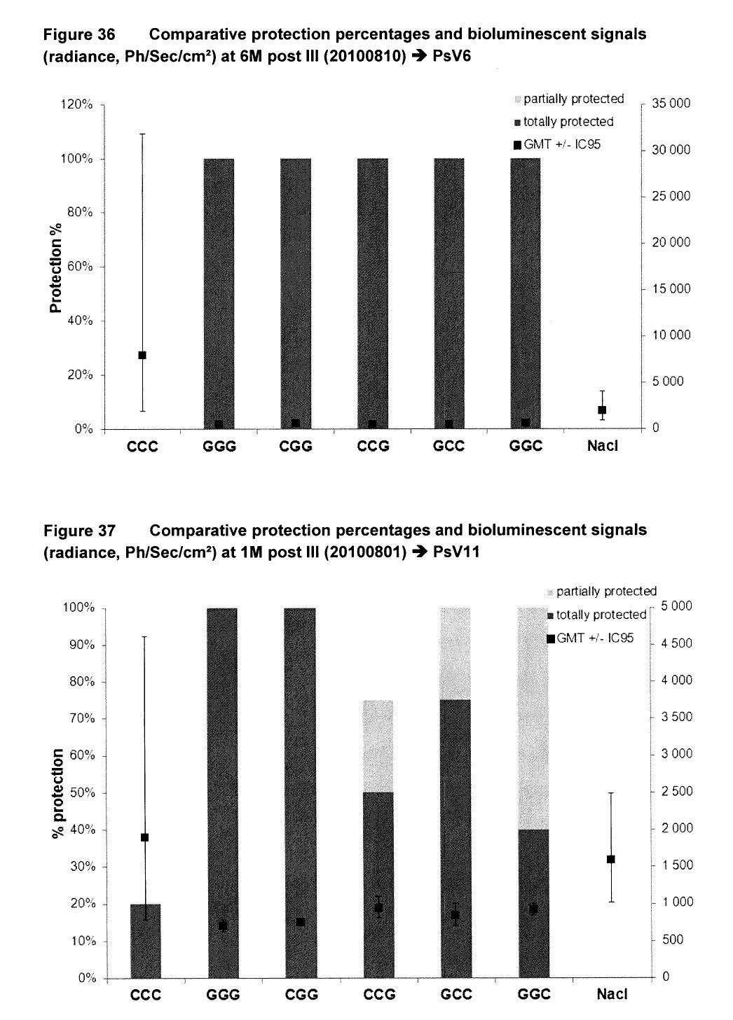

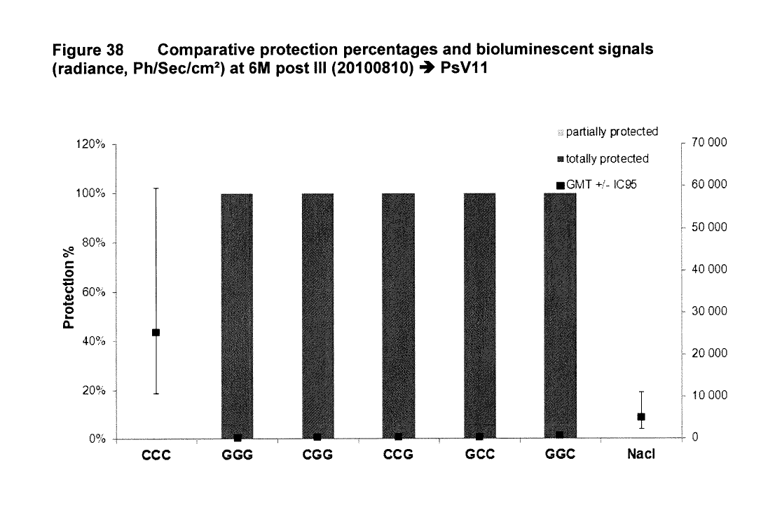

Comparison of protection percentages at 1M and 6M PIII following immunization with different vaccination schemes is presented in FIGS. 37 and 38 respectively.

| | CCC | GGG | CGG | CCG | GCC | GGC | NaCl |
|---|---|---|---|---|---|---|---|
| Protection % (M1 PIII) | 20% | 100% | 100% | 50 + | 75 + | 40 + | 0% |
| | | | | 25% | 25% | 60% | |

At 1 month post III 100% protection was observed with GGG and CGG
Good protection percentages were observed with CCG, GCC and GGC with a trend for better quality of protection with GCC
Low protection (20%) was observed with CCC

| | CCC | GGG | CGG | CCG | GCC | GGC | NaCl |
|---|---|---|---|---|---|---|---|
| Protection % (M6 PIII) | 0% | 100% | 100% | 100% | 100% | 100% | 0% |

At 6 months post III 100% protection was observed with GGG, CGG, CCG, GCC and GGC by contrast to CCC vaccination which was without any protection against PsV-11→GGG˜CGG˜CCG˜GCC˜GGC>CCC

Conclusions

Data generated show good persistent protection until 6 months post vaccination to PsV-18, 6 and 11 and confirm potential benefit of Cervarix™/Gardasil™ mixing. Intravaginal challenge mice model demonstrates similar conclusions to human for specific protection.

Correlation Protection Percentages and Total/Neutralizing Antibody Levels

The correlation between levels of total and neutralizing antibodies with protection percentages can be worked out to evaluate the minimal quantity of antibodies required to induce protection. Data are summarized in the table below.

Data 1 Month Post III

| | | CCC | GGG | CGG | CCG | GCC | GGC |
|---|---|---|---|---|---|---|---|
| HPV-18 | Protection % | Unavailable data | | | | | |
| | (total + partial) | | | | | | |
| | Total Abs | 154059 | 156657 | 122543 | 207997 | 112738 | 125080 |
| | (EU/ml) | | | | | | |
| | Nabs (ED50) | 120374 | 63182 | 103478 | 199974 | 164226 | 169196 |
| HPV-6 | Protection % | 0% | 100% | 100% | 100% | 100% | 100% |
| | (total + partial) | | | | | | |
| | Total Abs | 1438 | 39258 | 24508 | 12514 | 5903 | 16817 |
| | (EU/ml) | | | | | | |
| | Nabs (ED50) | <cut-off | 120557 | 113614 | 2531 | 2588 | 27728 |
| HPV-11 | Protection % | 20% | 100% | 100% | 50 + 25% | 75 + 25% | 40 + 60% |
| | (total + partial) | | | | | | |
| | Total Abs | 1028 | 39875 | 34488 | 14343 | 7484 | 23714 |
| | (EU/ml) | | | | | | |
| | Nabs (ED50) | <cut-off | 36651 | 79618 | 1532 | 2276 | 12379 |

Data 6 Months Post III

| | | CCC | GGG | CGG | CCG | GCC | GGC |
|---|---|---|---|---|---|---|---|
| HPV-18 | Protection % | 100% | 100% | 80% | 100% | 100% | 100% |
| | (total + partial) | | | | | | |
| | Total Abs (EU/ml) | 67637 | 68725 | 59129 | 101312 | 73996 | 51293 |
| | Nabs (ED50) | 74760 | 17801 | 56757 | 68843 | 50816 | 45063 |
| HPV-6 | Protection % | 0% | 100% | 100% | 100% | 100% | 100% |
| | (total + partial) | | | | | | |
| | Total Abs (EU/ml) | 1520 | 23106 | 8027 | 6158 | 3966 | 13317 |
| | Nabs (ED50) | <cut-off | 54513 | 29170 | 1284 | 1619 | 17808 |
| HPV-11 | Protection % | 0% | 100% | 100% | 100% | 100% | 100% |
| | (total + partial) | | | | | | |
| | Total Abs (EU/ml) | 1244 | 21346 | 9157 | 5910 | 4275 | 10842 |
| | Nabs (ED50) | <cut-off | 16153 | 8791 | 1413 | 2131 | 2645 |

Absence of protection to PsV-6 and PsV-11 with CCC seems to correlate with low levels of total anti-HPV6/11 responses and absence of neutralizing antibodies to HPV-6 and 11.

Overall Result Highlights for Example 3:

Immunogenicity
Total antibodies (ELISA)
   No impact of Cervarix™ on HPV-18 ELISA antibody responses at 1 and 6 months post vaccination→GGG˜GCC˜GGC
   Negative impact of Cervarix™ on HPV-6 ELISA: Cervarix not capable of boosting pre-existing HPV-6 responses at 1 month post III→GGG>GCC˜GGC
   Negative impact of Cervarix™ 2× on HPV-6 ELISA at 6 months post III but similar responses observed with GGG and GGC→GGG˜GGC>GCC
   Negative impact of Cervarix™ 2× on HPV-11 ELISA at 1 and 6 months post III but similar responses observed with GGG and GGC→GGG˜GGC>GCC
Neutralizing antibodies (PBNA)
   Similar neutralizing antibodies responses to HPV-18 observed with all vaccination schemes 1 month PIII, only lower responses with GGG compared to CCC 6 months post vaccination.
   Similar neutralizing antibodies responses to HPV-6 and 11 when Cervarix™ prime followed by 2 doses of Gardasil™ compared to classical Gardasil™ 3 doses.

Efficacy (Intravaginal Challenge Mice Model)
HPV-18: high persistent protection until 6 months post III with all 6 vaccination schemes

Overall Conclusions for Example 3

The added value of priming with Cervarix™ compared to a 3 dose vaccination scheme with Cervarix™ or Gardasil™ was not confirmed in this experiment. This could be linked to the vaccination schedule corresponding to D0145/120 compared to previous data observed with classical D0121/120 scheme. Notably, CCC did not perform in comparison to GGG as it does in the clinics (see Einstein et al 2009).

Vaccination with 1 or 2 doses of Cervarix™ in a 3 dose vaccination scheme shows 100% full protection to PsV-6 and 11 like a classical Gardasil™ 3 dose scheme. Moreover, high (80 to 100%) protection to PsV-18 was observed for usual Cervarix™ and Gardasil™ 3 doses schemes but it was also observed for groups injected with mixed vaccines. These data confirm a potential benefit of Cervarix™/Gardasil™ mixing.

No protection to PsV-6 and PsV-11 was observed after vaccination with 3 doses of Cervarix™, this correlates with clinical data and demonstrates relevance of the intravaginal challenge mice model in the context of specific and cross reactive responses to PsV-6/18 and 11.

Overall Conclusion for Examples 1, 2 and 3

Immunogenicity

Serological data demonstrated an added value of priming with Cervarix™ at least 1× (total and neutralizing HPV-16/18 responses) compared to a 3 dose vaccination scheme with Gardasil™. Total and neutralizing antibodies responses to HPV-11 were also higher when priming with 1 dose of Cervarix™ followed by 2 doses of Gardasil™ compared to classical Gardasil™ 3 doses. Added value priming with Cervarix™ (1 or 2 doses) compared to 3 doses of Gardasil™ was observed for total anti-HPV6 responses but not for neutralizing antibodies.

Compared to classical Cervarix™ 3 doses, priming with 1 (HPV-6 and 11) or 2 doses (HPV-16) of Cervarix™ induces higher total and neutralizing responses to HPV-16/6 and 11.

These data were observed in a 3 dose scheme with 1/10^{th} HD but were not confirmed with 1/50^{th} HD. This vaccine dilution was tested in a D0-45-120 scheme and data generated did not demonstrate higher anti-VLP18 responses with CCC compared to GGG as usual. Based on the fact that higher anti-VLP18 responses are maintained for Cervarix™ in a 2 doses scheme with 1/50^{th} HD, D0-45-120 vaccination schedule does not seem to be optimal for this evaluation.

The added value of priming with 1 dose of Cervarix™ is maintained in a 2 dose scheme with 1/50^{th} HD by demonstrating higher total anti-HPV18 responses and similar total anti-HPV11 responses compared to 2 doses of Gardasil™.

Efficacy

Efficacy data were generated after vaccination with 3 doses (CCC, GGG, CCG, CGG, GCC or GGC) or 2 doses (CC, GG, CG or GC) with 1/50^{th} HD for each vaccine.

Specific protection against PsV-18 was demonstrated with all 3 dose vaccination schemes until 6 months post III. Moreover, 100% protection to PsV-6 and PsV-11 was demonstrated with classical 3 doses Gardasil™ but also with GCC, GGC, CGG and CCG and this was sustained until 6 mo [symbol] ination. As expected, no cross-protection to PsV-6 and PsV-11 was observed after vaccination with 3 doses of Cervarix™.

Surprisingly, 100% protection to PsV-11 was also reached after vaccination with CG 1/50^{th} HD without any neutralizing antibody responses despite high levels of ELISA titers induced. As for a 3 dose vaccination scheme, no cross-protection against PsV-11 was observed with CC.

These data demonstrate the potential to mix Cervarix™/Gardasil™ vaccines by maintaining high level of protection to specific types (HPV-18/6/11). CG, CCG and CGG immunisation schemes could be good candidates by combining protection against high risk HPV types and genital warts.

1-2. (canceled)

3. A method for the prevention of HPV infection or disease in an individual, which method comprises:
(i) administering to the individual at least one dose of a first immunogenic composition comprising HPV VLPs from one or more HPV types in combination with an adjuvant comprising a TLR agonist; and
(ii) administering to the individual at least one dose of a second immunogenic composition comprising HPV VLPs from one or more HPV types which second immunogenic composition does not comprise a TLR agonist;
wherein the first immunogenic composition increases at least one of a type specific immune response or cross-reactive immune response to a type present in the second immunogenic composition, which is not present in the first immunogenic composition.

4. The method of claim 3 wherein the first immunogenic composition comprises HPV 16 or HPV 18 VLPs, or HPV 16 and HPV 18 VLPs.

5. The method according to claim 3 wherein the first immunogenic composition increases the type specific immune response to HPV 16 or HPV 18 or both HPV 16 and HPV 18.

6. The method of claim 3 wherein the first immunogenic composition increases the type specific immune response compared to the immune response to that HPV type when an equivalent number of doses of only the second immunogenic composition are administered.

7. The method of claim 3 wherein the first immunogenic composition generates a cross reactive immune response against one or more high risk or low risk HPV types present in the second immunogenic composition.

8. (canceled)

9. The method of claim 3 wherein the first immunogenic composition generates a cross reactive immune response against HPV 6 and the second immunogenic composition comprises HPV 6 VLPs.

10. The method of claim 3 wherein the first immunogenic composition generates a cross reactive immune response against HPV 11 and the second immunogenic composition comprises HPV 11 VLPs.

11. (canceled)

12. The method of claim 3 wherein the second immunogenic composition comprises HPV 6, 11, 16, and 18 VLPs and optionally other types.

13. (canceled)

14. The method of claim 3 wherein the first immunogenic composition comprises a TLR4 agonist.

15. The method of claim 14 wherein the TLR4 agonist is MPL.

16-17. (canceled)

18. The method of claim 3 wherein the second immunogenic composition comprises an aluminium salt.

19. (canceled)

20. The method of claim 3 wherein two doses of the first immunogenic composition are administered followed by one or more doses of the second immunogenic composition.

21. The method of claim 3 wherein one dose of the first immunogenic composition is administered followed by one or two or more doses of the second immunogenic composition.

22-23. (canceled)

24. The method of claim 3 wherein the HPV VLPs comprise L1 or an immunogenic fragment thereof.

25. (canceled)

26. A kit comprising:
(i) a first immunogenic composition comprising VLPs from at least one HPV type in combination with an adjuvant comprising a TLR agonist; and
(ii) a second immunogenic composition comprising VLPs from at least one HPV type, which second immunogenic composition does not comprise a TLR agonist.

27. The kit of claim 26 wherein the first and second immunogenic compositions comprise HPV 16 and HPV 18 VLPs.

28. The kit of claim 26 wherein the second immunogenic composition further comprises HPV 6 and/or HPV 11 VLPs which are absent from the first immunogenic composition.

29. (canceled)

30. The kit of claim 26 wherein the first immunogenic composition comprises a TLR4 agonist.

31. The kit of claim 30 wherein the TLR4 agonist is MPL.

32. (canceled)

33. The kit of claim 26 wherein the second immunogenic composition comprises an aluminium salt.

34. The kit of claim 33 wherein the first immunogenic composition comprises aluminium hydroxide and the second immunogenic composition comprises aluminium hydroxyphosphate sulphate.
